(19) United States
(12) Patent Application Publication
(10) Pub. No.: US 2024/0157544 A1
(43) Pub. Date: May 16, 2024

(54) OVERSUIT WITH STATIC POSITION SUPPORT

(71) Applicant: Seismic Holdings, Inc., Menlo Park, CA (US)

(72) Inventors: Masood Nevisipour, Scottsdale, AZ (US); Ryan Henry, Beaverton, OR (US); Alexander Jonas Band, Alameda, CA (US); Ray Cowan, Mountain View, CA (US); Michael Todaro, Santa Clara, CA (US); Jesus Antonio Prado de la Mora, Foster City, CA (US)

(21) Appl. No.: 18/328,747

(22) Filed: Jun. 4, 2023

Related U.S. Application Data
(60) Provisional application No. 63/349,098, filed on Jun. 5, 2022.

(51) Int. Cl. B25J 9/00; A61B 5/00; A61H 3/00

(52) U.S. Cl. CPC B25J 9/0006; A61B 5/6804; A61H 3/00; A61B 2562/0219; A61H 2003/007

(57) ABSTRACT

An oversuit system and method are provided for assisting a user of the oversuit in maintaining a relatively static position.

### TECHNICAL FIELD

This disclosure relates generally to oversuits, and more specifically to oversuits that aid users engaged in relatively static positions.

### BACKGROUND

Wearable robotic systems have been developed for augmentation of humans' natural capabilities, or to replace functionality lost due to injury or illness.

### SUMMARY

An oversuit system and method for the use thereof are provided for assisting a user of in maintaining a relatively static position.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIGS. 1A-1C show front, back, and side views of a human female, in particular showing potential locations on the body where load distribution members (LDMs) of an oversuit can be positioned according to an embodiment.

FIGS. 1D-1F show front, back, and side views, respectively, of a power layer according to an embodiment.

FIGS. 1G and 1H show respective front and back views of a human male's musculature anatomy, according to an embodiment.

FIGS. 11 and 1J show front and side views of an illustrative oversuit having several power layer segments that approximate many of the muscles shown in FIGS. 1G and 1H, according to various embodiments.

FIGS. 2A, 2B, and 2C show front, side, and back views of an illustrative oversuit according to an embodiment.

FIGS. 2D-2F show front, side, and back views of an illustrative oversuit with the belt removed according to an embodiment.

FIGS. 2G, 2H, and 2I show front, side, and back views of an alternative of oversuit according to an embodiment.

FIGS. 2J, 2K, and 2L illustrate another embodiment of and oversuit according to an embodiment.

FIG. 3 shows a detailed front view of a knee load distribution member according to an embodiment.

FIG. 3A shows a detailed front view of another knee load distribution member according to an embodiment.

FIG. 4 shows a more detailed view of a belt according to an embodiment.

FIGS. 4A, 4B, 4C and 4D shows detailed views of another belt embodiment.

FIGS. 4E, and 4F illustrate hip pouches that can be used on conjunction with a belt according to an embodiment.

FIG. 5 shows an illustrative symbiosis oversuit system according to an embodiment.

FIG. 6 shows illustrative process for implementing a symbiosis oversuit system according to an embodiment.

FIGS. 7A, 7B, 7C, and 7D show illustrative lines of actions provided by an oversuit according to an embodiment.

FIG. 8 shows an illustrative re-route structure according to an embodiment.

FIG. 9 shows an illustrative chart of which lines of action are used for a particular static position according to an embodiment.

FIG. 10 show a knee sensor enclosure according to an embodiment.

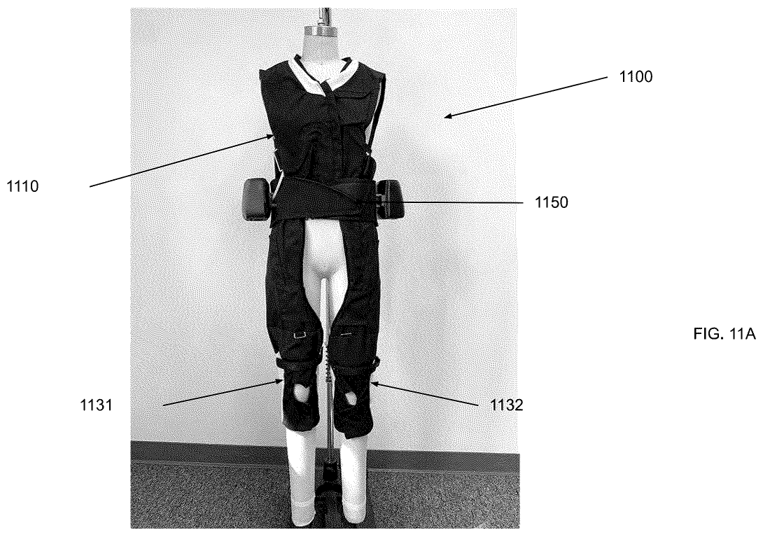

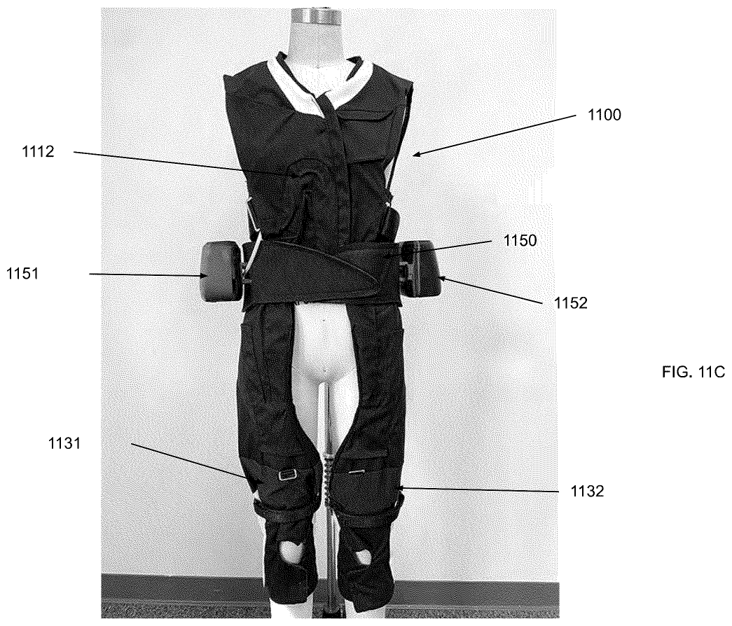

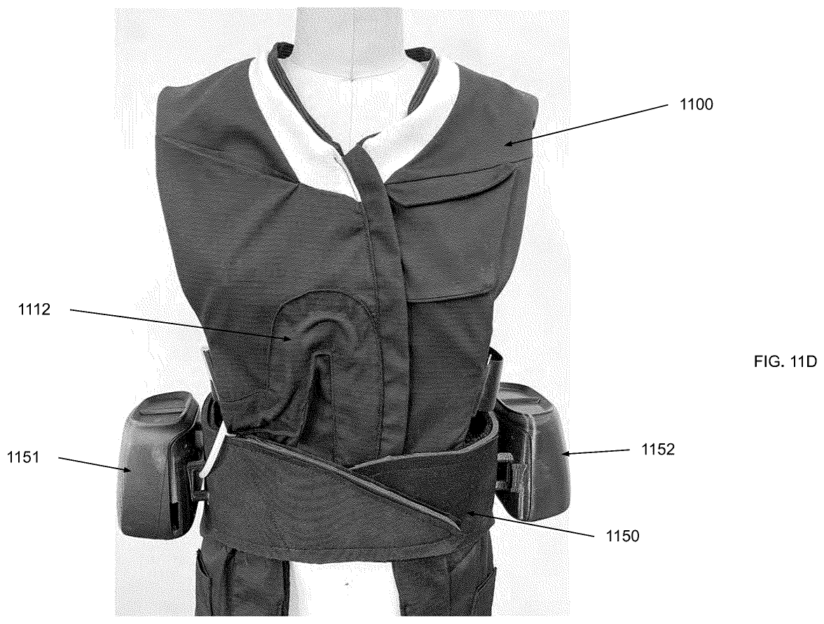

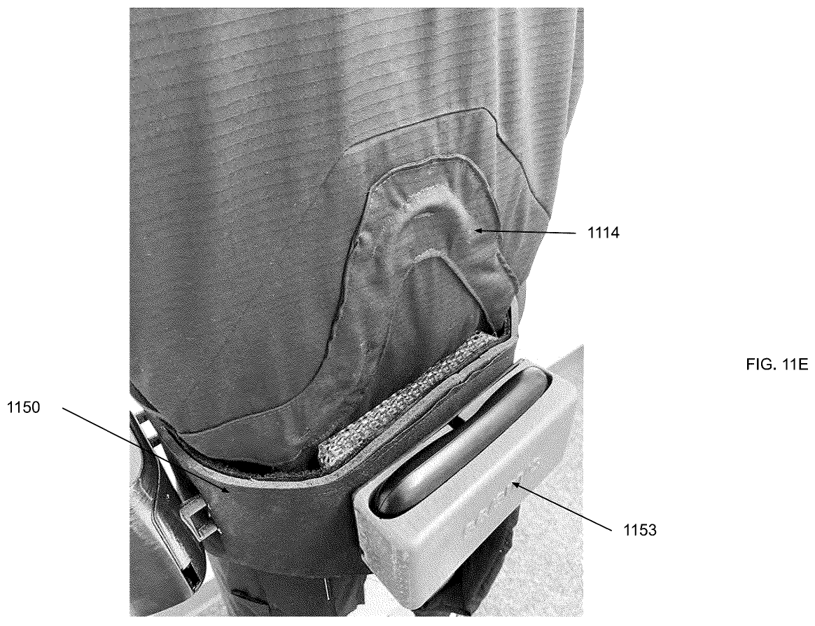

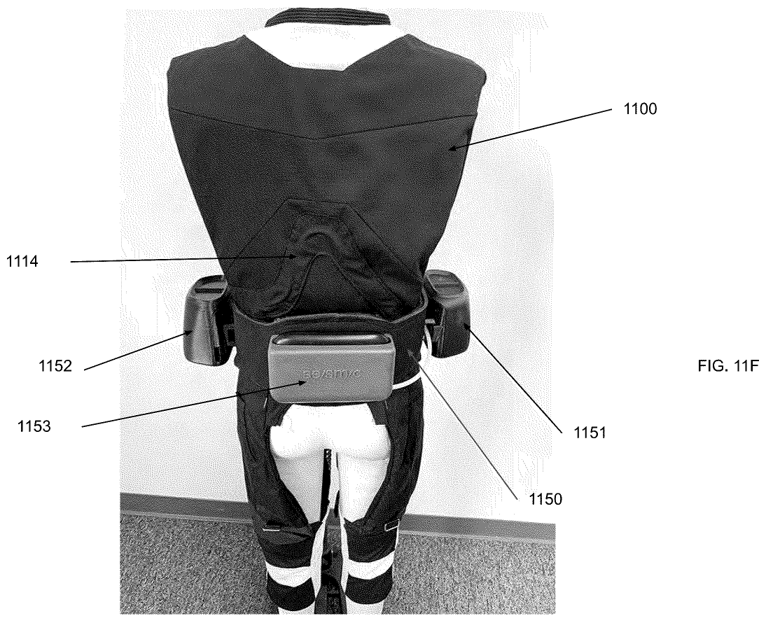

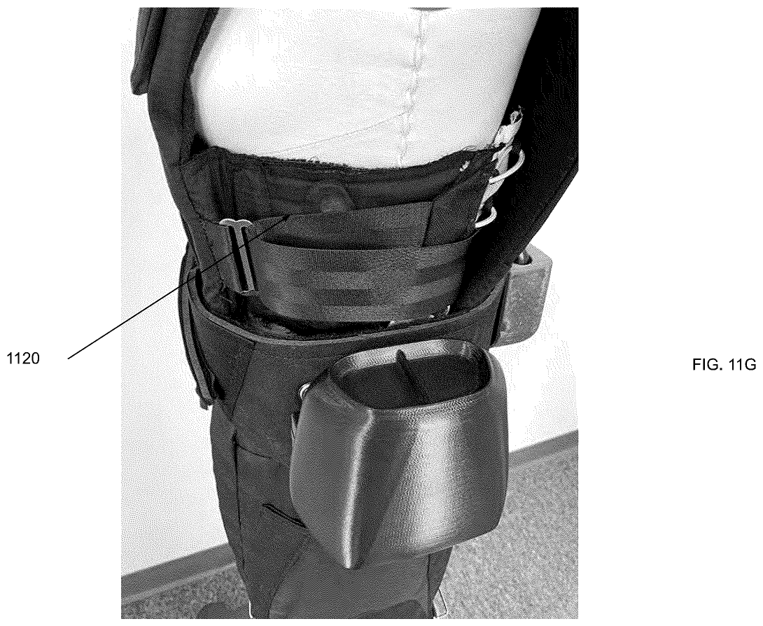

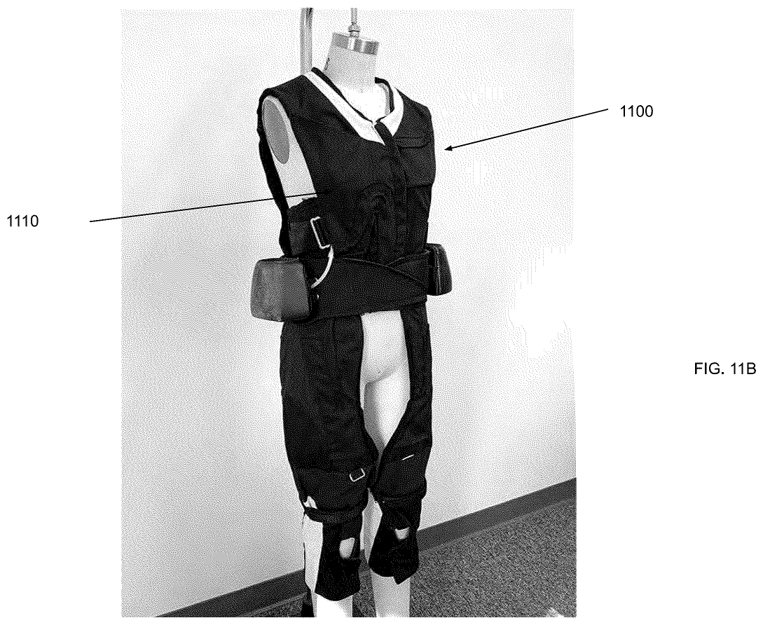

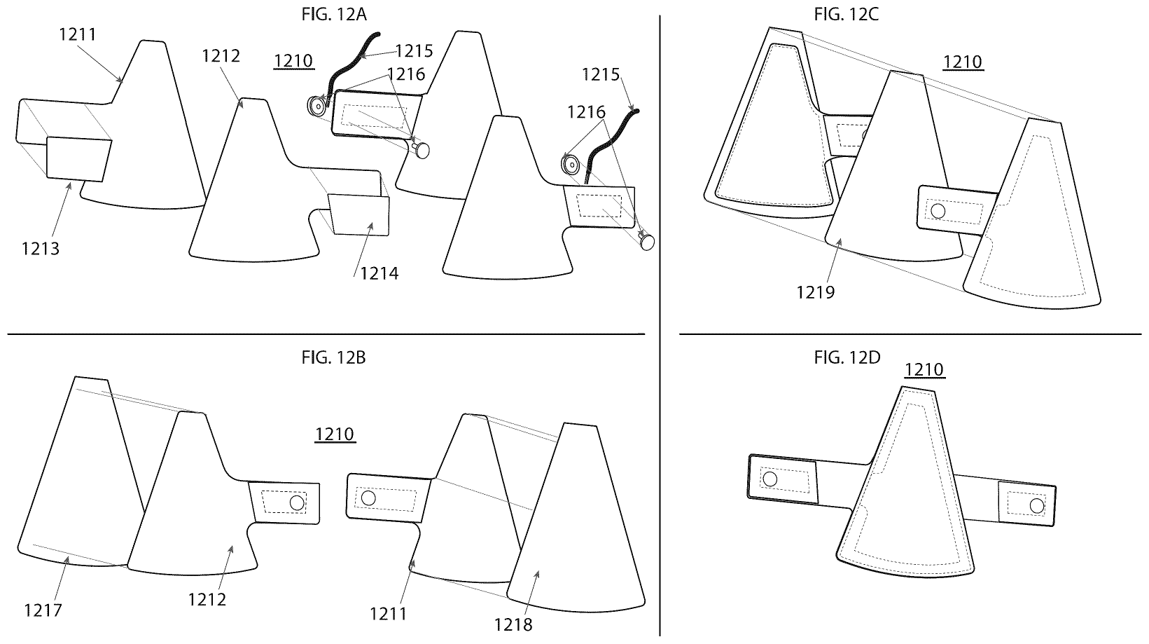

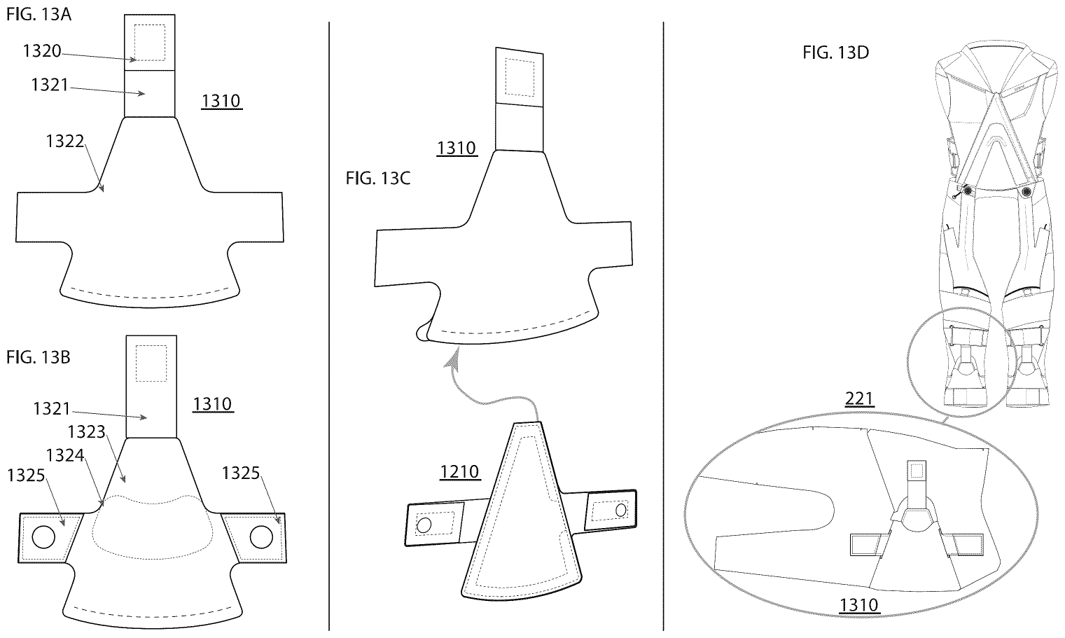

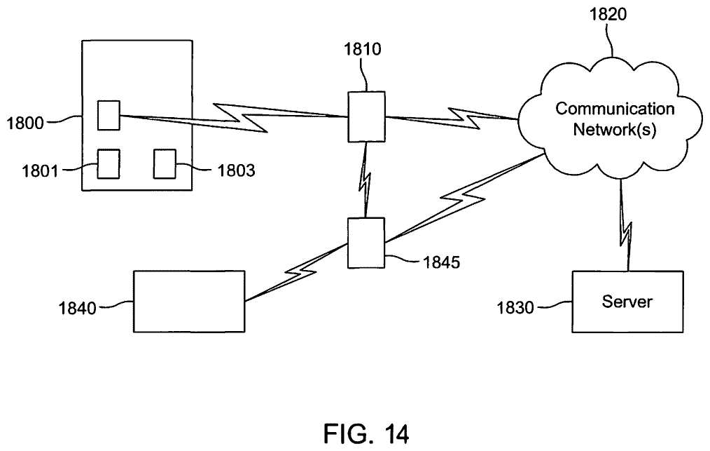

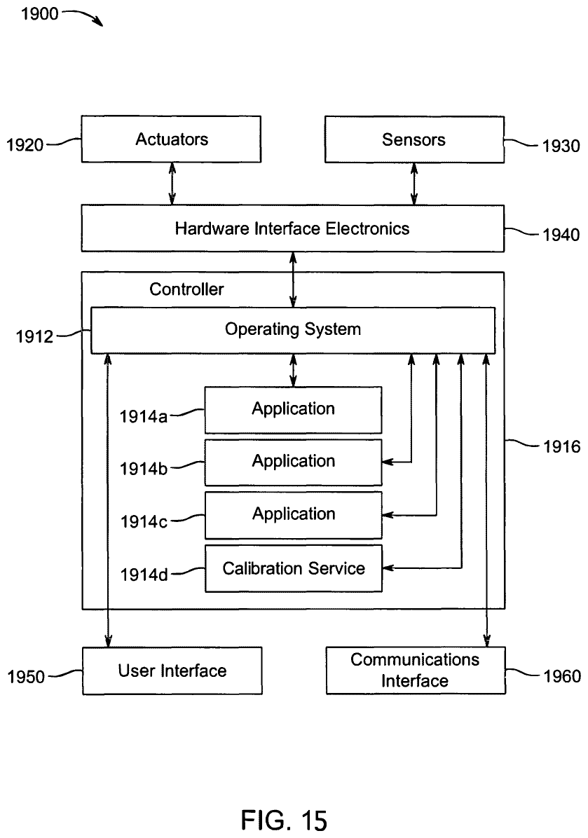

FIGS. 11A-11G show different views of an oversuit according to an embodiment.

FIGS. 12A-12D show a knee sensor according to an embodiment.

FIGS. 13A-13D show containment and attachment for a soft knee sensor assembly according to an embodiment.

FIG. 14 illustrates an example oversuit according to an embodiment.

FIG. 15 is a schematic illustrating elements of an oversuit and a hierarchy of control or operating the oversuit according to an embodiment.

### DETAILED DESCRIPTION

In the following description, numerous specific details are set forth regarding the systems, methods and media of the disclosed subject matter and the environment in which such systems, methods and media may operate, etc., in order to provide a thorough understanding of the disclosed subject matter. It can be apparent to one skilled in the art, however, that the disclosed subject matter may be practiced without such specific details, and that certain features, which are well known in the art, are not described in detail in order to avoid complication of the disclosed subject matter. In addition, it can be understood that the examples provided below are exemplary, and that it is contemplated that there are other systems, methods and media that are within the scope of the disclosed subject matter.

In the descriptions that follow, an oversuit or assistive oversuit is a suit that is worn by a wearer over the wearer's normal clothing. The oversuit may be supportive and/or assistive, as it physically supports or assists the wearer in performing particular activities, or can provide other functionality such as communication to the wearer through physical expressions to the body, engagement of the environment, or capturing of information from the wearer. In some embodiments, the oversuit can include one or more suit components designed to be worn on specific locations of the wearer and can also include several subsystems. In some embodiments, the oversuit can be outfitted with the appropriate suit components and subsystems depending on the intended use by the end user. The suit components can include, for example, a vest component, a belt component, and leg component. The suit components can include load distribution members that provide a platform for tensioning elements to effectively impart movement or stability assistance to the user of the oversuit. The subsystems can include components that are integrated within or attached to suit components. The subsystem components can include, for example, motors, one or more batteries, sensors, tension strings, string routing conduits, power and signal routing conduits, processors, communications circuitry, user interface controls, etc. Some of these subsystems components are referred to herein as the power layer.

The suit components can provide the interfaces between the oversuit and the wearer's body. The suit components may be adapted to be worn directly against the wearer's skin, between undergarments and outer layers of clothing, over outer layers of clothing or a combination thereof, or the suit components may be designed to be worn as primary clothing itself. In some embodiments, the suit components can be adapted to be both comfortable and unobtrusive, easy to don and doff, comfortably and efficiently transmit loads to the wearer's body via integrated load distribution members to provide movement or stability assistance, and to secure and route power and signal conductors and tensioning components. The suit components can include several different material types. Elastic materials may provide compliance to conform to the wearer's body and allow for ranges of movement. Substantially inextensible materials may be used to transfer loads from the power layer to the wearer's body. These materials may be substantially inextensible in one axis, yet flexible or extensible in other axes such that the load transmission is along preferred paths. The load transmission paths may be optimized to distribute the loads across regions of the wearer's body to minimize the forces felt by the wearer, while providing efficient load transfer with minimal loss and not causing the load distribution members to slip (or ride up on the wearer's clothing).

In one embodiment, the load distribution members may incorporate one or more catenary curves to distribute loads across the wearer's body. Multiple load distribution members or catenary curves may be joined with pivot points, such that as loads are applied to the structure, the arrangement of the load distribution members pivots, tightens or constricts on the body to increase the gripping strength. Compressive elements such as battens, rods, or stays may be used to transfer loads to different areas of the base layer for comfort or structural purposes. For example, a power layer component may terminate in the middle back due to its size and orientation requirements, however the load distribution members that anchor the power layer component may reside on the lower back. In this case, one or more compressive elements may transfer the load from the power layer component at the middle back to the load distribution member at the lower back. In another embodiment, any load distribution member may have multiple attachment points for connection to the power layer. The use of multiple attachment points can distribute a tensioning load provided by the power layer across the load distribution member, thereby increasing load transfer efficiency and comfort for the wearer.

The load distribution members may be constructed using multiple fabrication and textile application techniques. For example, the load distribution member can be constructed from a layered woven 45°/90° with bonded edge, spandex tooth, organza (poly) woven 45°/90° with bonded edge, organza (cotton/silk) woven 45°/90°, and Tyvek (non-woven). The load distribution member may be constructed using knit and lacing or horse hair and spandex tooth. The load distribution member may be constructed using channels and/or laces.

The load distribution member may include a flexible underlayer that is constructed to compress against a portion of the wearer's body, either directly to the skin, or to a clothing layer. The load distribution members facilitate transmission of shears or other forces from the a body segment (via clothing worn over the body segment) to maintain force trajectories of the members relative to such a body segment, or to provide some other functionality. Such a flexible underlayer can have a flexibility and/or compliance that differs from other layers of the load distribution member (e.g., that is less than that of the other layer(s), at least in a direction along the members), such that the load distribution member can transmit forces along their length and evenly distribute shear forces and/or pressures a body segment to which load distribution member is mounted.

Further, such a flexible underlayer can be configured to provide additional functionality. The material of the flexible underlayer could include anti-bacterial, anti-fungal, or other agents (e.g., silver nanoparticles) to prevent the growth of microorganisms. The flexible underlayer can be configured to manage the transport of heat and/or moisture (e.g., sweat) from a wearer to improve the comfort and efficiency of activity of the wearer. The flexible underlayer can include straps, seams, hook-and-loop fasteners, clasps, zippers, or other elements configured to maintain a specified relationship between elements of the load distribution members and aspects of a wearer's anatomy. The underlayer can additionally increase the ease with which a wearer can don and/or doff the flexible body harness and/or a system or garment that includes the flexible body harness. The underlayer can additionally be configured to protect the wearer from ballistic weapons, sharp edges, shrapnel, or other environmental hazards (by including, e.g., panels or flexible elements of para-aramid or other high-strength materials).

The load distribution member can additionally include features such as size adjustments, openings and electro-mechanical integration features to improve ease of use and comfort for the wearer.

Size adjustment features permit the oversuit to be adjusted to the wearer's body. The size adjustments may allow the suit to be tightened or loosened about the length or circumference of the torso or limbs. The adjustments may comprise lacing, the Boa system, webbing, elastic, buckles, bindings such as SPD® bindings, hook-and-loop or other fasteners. Size adjustment may be accomplished by the load distribution members themselves, as they constrict onto the wearer when loaded. In one example, the torso circumference may be tightened with corset-style lacing, the legs tightened with hook-and-loop in a double-back configuration, and the length and shoulder height adjusted with webbing and tension-lock fasteners such as cam-locks, D-rings or the like. The size adjustment features in the load distribution members may be actuated by the power layer to dynamically adjust the base layer to the wearer's body in different positions, in order to maintain consistent pressure and comfort for the wearer. For example, the load distribution members (e.g., positioned around or near the knees) may be required to tighten when standing, and loosen when sitting such that the load distribution member does not excessively constrict body parts when seated. The dynamic size adjustment may be controlled, for example, by detecting pressures or forces in one or more load distribution members and actuating the power layer to consistently attain the desired force or pressure. This feature does not necessarily cause the suit to provide physical assistance, but can create a more comfortable experience for the wearer, or allow the physical assistance elements of the suit to perform better or differently depending on the purpose of the movement assistance.

Opening features in the suit components facilitate donning (putting the oversuit on) and doffing (taking the oversuit off) for the wearer. Opening features may comprise zippers, hook-and-loop, snaps, buttons or other textile fasteners. In one example, a front, central zipper provides an opening feature for the torso, while hook-and-loop fasteners provide opening features for the legs and shoulders. In this case, the hook-and-loop fasteners provide both opening and adjustment features. In other examples, the oversuit may simply have large openings, for example around the arms or neck, and elastic panels that allow the suit to be donned and doffed without specific closure mechanisms. A truncated load distribution member may be simply extended to tighten on the wearer's body. Openings may be built into the oversuit such that no fabric or power layers requires removal when the user needs to use the bathroom.

Electro-mechanical integration features for attaching subsystem components together can be used to facilitate removal/installation of power layer components and for donning and doffing the oversuit. For example, a belt load distribution member may be donned by the wearer after the suit components have been donned. After the belt load distribution member is donned, the user may then connect belt components to suit components. For example, the motors located on the belt load distribution member can be connected to tensioning members via quick disconnects.

Electromechanical integration features may also protect or cosmetically hide various components. For example, tensioning elements such as twisted strings may be routed through sleeves, tubes, or channels integrated into the suit components which can both conceal and protect these components. The sleeves, tubes, or channels may also permit motion of the component, for example during actuation of a power layer element. The sleeves, channels, or tubes may comprise resistance to collapse, ensuring that the component remains free and uninhibited within. In addition, signal and power routing cables may be routed through sleeves, tubes, or channels existing in the suit components. In some embodiments the tensioning members and signal and power routing cables may share the same sleeves, tubes, and channels.

Enclosures, padding, fabric coverings, or the like may be used to further integrate components into the suit components for cosmetic, comfort, or protective purposes. For example, components such as motors, batteries, cables, or circuit boards may be housed within an enclosure, fully or partially covered or surrounded in padded material such that the components do not cause discomfort to the wearer, are visually unobtrusive and integrated into the oversuit, and are protected from the environment. Opening and closing features may additionally provide access to these components for service, removal, or replacement.

Electro-mechanical integration features may also include wireless communication. Rather than utilizing physical electrical connections to power layer components such as sensors and motors, a processor can communicate with the one or more power layer components via wireless communication protocols such as Bluetooth, ZigBee, ultrawide band, or any other suitable communication protocol. This may reduce the electrical interconnections required within the suit. Each of the one or more power layer components may additionally incorporate a local battery such that each power layer component or group of power layer components are independently powered units that do not require direct electrical interconnections to other areas of the oversuit.

In some embodiments, the power layer may include stability elements provide passive mechanical stability and assistance to the wearer. The stability elements can include passive (non-powered) spring or elastic elements that generate forces or store energy to provide stability or assistance to the wearer. An elastic element can have an un-deformed, least-energy state. Deformation, e.g. elongation, of the elastic element stores energy and generates a force oriented to return the elastic element toward its least-energy state. For example, elastic elements approximating hip flexors and hip extensors may provide stability to the wearer in a standing position. As the wearer deviates from the standing position, the elastic elements are deformed, generating forces that stabilize the wearer and assist maintaining the standing position. In another example, as a wearer moves from a standing to seated posture, energy is stored in one or more elastic elements, generating a restorative force to assist the wearer when moving from the seated to standing position. Similar passive, elastic elements may be adapted to the torso or other areas of the limbs to provide positional stability or assistance moving to a position where the elastic elements are in their least-energy state.

Elastic elements of the stability layer may be integrated to parts of a load distribution member or be an integral part of the power layer. For example, elastic fabrics containing spandex or similar materials may be used. Elastic elements may also include discrete components such as springs or segments of elastic material such as silicone or elastic webbing, anchored to the base layer for load transmission at discrete points, as described above.

The stability elements may be adjusted as described above, both to adapt to the wearer's size and individual anatomy, as well as to achieve a desired amount of pre-tension or slack in components of the stability layer in specific positions. For example, some wearers may prefer more pre-tension to provide additional stability in the standing posture, while others may prefer more slack, so that the passive layer does not interfere with other activities such as ambulation.

The stability elements may interface with the power layer to engage, disengage, or adjust the tension or slack in one or more elastic elements. In one example, when the wearer is in a standing position, the power layer may pre-tension one or more elastic elements of the stability layer to a desired amount for maintaining stability in that position. The pre-tension may be further adjusted by the power layer for different positions or activities. In some embodiments, the elastic elements of the stability layer should be able to generate at least 5 lbs force; preferably at least 50 lbs force when elongated.

The power layer can provide active, powered assistance to the wearer, as well as electromechanical clutching to maintain components of the power or stability layers in a desired position or tension. The power layer can include one or more flexible linear actuators (FLA). An FLA is a powered actuator capable of generating a tensile force between two attachment points, over a give stroke length. An FLA is flexible, such that it can follow a contour, for example around a body surface, and therefore the forces at the attachment points are not necessarily aligned. In some embodiments, one or more FLAs can include one or more twisted string actuators. In the descriptions that follow, FLA refers to a flexible linear actuator that exerts a tensile force, contracts or shortens when actuated. The FLA may be used in conjunction with a mechanical clutch that locks the tension force generated by the FLA in place so that the FLA motor does not have to consume power to maintain the desired tension force. Examples of such mechanical clutches are discussed below. In some embodiments, FLAs can include one or more twisted string actuators or flexdrives, as described in further detail in U.S. Pat. No. 9,266,233, titled “Oversuit System,” the contents of which are incorporated herein by reference. FLAs may also be used in connection with electrolaminate clutches, which are also described in the U.S. Pat. No. 9,266,233. The electrolaminate clutch (e.g., clutches configured to use electrostatic attraction to generate controllable forces between clutching elements) may provide power savings by locking a tension force without requiring the FLA to maintain the same force.

The powered actuators, or FLAs, are arranged on different points on the body, to generate forces for assistance with various activities. The arrangement can often approximate the wearer's muscles, in order to naturally mimic and assist the wearer's own capabilities. For example, one or more FLAs may connect the back of the torso to the back of the legs, thus approximating the wearer's hip extensor muscles. Actuators approximating the hip extensors may assist with activities such as standing from a seated position, sitting from a standing position, walking, or lifting. Similarly, one or more actuators may be arranged approximating other muscle groups, such as the hip flexors, spinal extensors, abdominal muscles or muscles of the arms or legs.

The one or more FLAs approximating a group of muscles are capable of generating at least 10 lbs over at least a ½ inch stroke length within 4 seconds. In some embodiments, one or more FLAs approximating a group of muscles may be capable of generating at least 250 lbs over a 6-inch stroke within ½ second. Multiple FLAs, arranged in series or parallel, may be used to approximate a single group of muscles, with the size, length, power, and strength of the FLAs optimized for the group of muscles and activities for which they are utilized.

Power layer embodiments discussed herein use multi-joint actuation systems and tensioning systems. Multi-joint actuation systems can apply forces across two or more joints (e.g., a lumbar and hips) using a single drive chain. This is in contrast with a single joint actuation system in which a drive chain applies a force across only one joint. Thus, if force is to be applied across two joints, for example, two different single joint actuation system may be required, one for the first joint, and another for the second joint. This actuation system per joint requirement is eliminated with the multi-joint actuation system because one drive chain can apply forces to multiple joints, thereby simplifying and reducing the number of components needed to apply forces to those joints.

Power layer embodiments discussed herein can use a tensioning system. The tensioning system can provide “muscle” and “tendon” action of assistance movements. The “muscle” action provides the contraction in the form of shortening twisted string and the “tendon” action applies the assistive force to the user of the oversuit. The tensioning system can include tunnels and re-routing members to provide a secure pathway for the twisted strings within the fabric of the suit.

The sensor and controls layer captures data from the suit and wearer, utilizes the sensor data and other commands to control the power layer based on the activity being performed, and provides suit and wearer data to the UX/UI layer for control and informational purposes.

Sensors such as encoders or potentiometers may measure the length and rotation of the FLAs, while force sensors measure the forces applied by the FLAs. Inertial measurement units (IMUs) measure and enable computation of kinematic data (positions, velocities and accelerations) of points on the suit and wearer. These data enable inverse dynamics calculations of kinetic information (forces, torques) of the suit and wearer. Electromyographic (EMG) sensors may detect the wearer's muscle activity in specific muscle groups. Electronic control systems (ECSs) on the suit may use parameters measured by the sensor layer to control the power layer. Data from the IMUs may indicate both the activity being performed, as well as the speed and intensity. For example, a pattern of IMU or EMG data may enable the ECS to detect that the wearer is walking at a specific pace. This information then enables the ECS, utilizing the sensor data, to control the power layer in order to provide the appropriate assistance to the wearer. Stretchable sensors may be used as a strain gauge to measure the strain of the elements in the stability layer, and thereby predict the forces in the elastic elements of the stability layer. Stretchable sensors may be embedded in the base layer or grip layer and used to measure the motion of the fabrics in the base layer and the motion of the body.

Data from the sensor layer may be further provided to the UX/UI layer, for feedback and information to the wearer, caregivers or service providers.

The UX/UI layer comprises the wearer's and others' interaction and experience with the oversuit system. This layer includes controls of the suit itself such as initiation of activities, as well as feedback to the wearer and caregivers. A retail or service experience may include steps of fitting, calibration, training and maintenance of the oversuit system. Other UX/UI features may include additional lifestyle features such as electronic security, identity protection and health status monitoring.

The assistive oversuit can have a user interface for the wearer to instruct the suit which activity is to be performed, as well as the timing of the activity. In one example, a user may manually instruct the oversuit to enter an activity mode via one or more buttons, a keypad, or a tethered device such as a mobile phone. In another example, the oversuit may detect initiation of an activity from the sensor and controls layer, as described previously. In yet another example, the user may speak a desired activity mode to the suit, which can interpret the spoken request to set the desired mode. The suit may be pre-programmed to perform the activity for a specific duration, until another command is received from the wearer, or until the suit detects that the wearer has ceased the activity. The suit may include cease activity features that, when activated, cause the suit to cease all activity. The cease activity features can take into account the motion being performed, and can disengage in a way that takes into account the user's position and motion, and safely returns the user to an unloaded state in a safe posture.

The oversuit may have a UX/UI controller that is defined as a node on another user device, such as a computer or mobile smart phone. The oversuit may also be the base for other accessories. For example, the oversuit may include a cell phone chip so that the suit may be capable of receiving both data and voice commands directly similar to a cell phone, and can communicate information and voice signals through such a node. The oversuit control architecture can be configured to allow for other devices to be added as accessories to the oversuit. For example, a video screen may be connected to the oversuit to show images that are related to the use of the suit. The oversuit may be used to interact with smart household devices such as door locks or can be used to turn on smart televisions and adjust channels and other settings. In these modes, the physical assist of the suit can be used to augment or create physical or haptic experiences for the wearer that are related to communication with these devices. For instance, an email could have a pat on the back as a form of physical emoji that when inserted in the email causes the suit to physically tap the wearer or perform some other type of physical expression to the user that adds emphasis to the written email.

The oversuit may provide visual, audio, or haptic feedback or cues to inform the user of various oversuit operations. For example, the oversuit may include vibration motors to provide haptic feedback. As a specific example, two haptic motors may be positioned near the front hip bones to inform the user of suit activity when performing a sit-to-stand assistive movement. In addition, two haptic motors may be positioned near the back hip bones to inform the user of suit activity when performing a stand-to-sit assistive movement. The oversuit may include one or more light emitting diodes (LEDs) to provide visual feedback or cues. For example, LEDS may be placed near the left and/or right shoulders within the peripheral vision of the user. The oversuit may include a speaker or buzzer to provide audio feedback or cues.

In other instances, the interaction of the FLA's with the body through the body harness and otherwise can be used as a form of haptic feedback to the wearer, where changes in the timing of the contraction of the FLA's can indicate certain information to the wearer. For instance, the number or strength of tugs of the FLA on the waist could indicate the amount of battery life remaining or that the suit has entered a ready state for an impending motion.

The control of the oversuit may also be linked to the sensors that are measuring the movement of the wearer, or other sensors, for instance on the suit of another person, or sensors in the environment. The motor commands described herein may all be activated or modified by this sensor information. In this example, the suit can exhibit its own reflexes such that the wearer, through intentional or unintentional motions, cues the motion profile of the suit. When sitting, for further example, the physical movement of leaning forward in the chair, as if to indicate an intention to stand up, can be sensed by the suit IMU's and be used to trigger the sit to stand motion profile. In one embodiment, the oversuit may include sensors (e.g., electroencephalograph (EEG) sensor) that are able to monitor brain activity may be used to detect a user's desire to perform a particular movement. For example, if the user is sitting down, the EEG sensor may sense the user's desire to stand up and cause the oversuit to prime itself to assist the user in a sit-to-stand assistive movement.

The suit may make sounds or provide other feedback, for instance through quick movements of the motors, as information to the user that the suit has received a command or to describe to the user that a particular motion profile can be applied. In the above reflex control example, the suit may provide a high pitch sound and/or a vibration to the wearer to indicate that it is about to start the movement. This information can help the user to be ready for the suit movements, improving performance and safety. Many types of cues are possible for all movements of the suit.

Control of the suit includes the use of machine learning techniques to measure movement performance across many instances of one or of many wearers of suits connected via the internet, where the calculation of the best control motion for optimizing performance and improving safety for any one user is based on the aggregate information in all or a subset of the wearers of the suit. The machine learning techniques can be used to provide user specific customization for oversuit assistive movements. For example, a particular user may have an abnormal gait (e.g., due to a car accident) and thus is unable to take even strides. The machine learning may detect this abnormal gait and compensate accordingly for it.

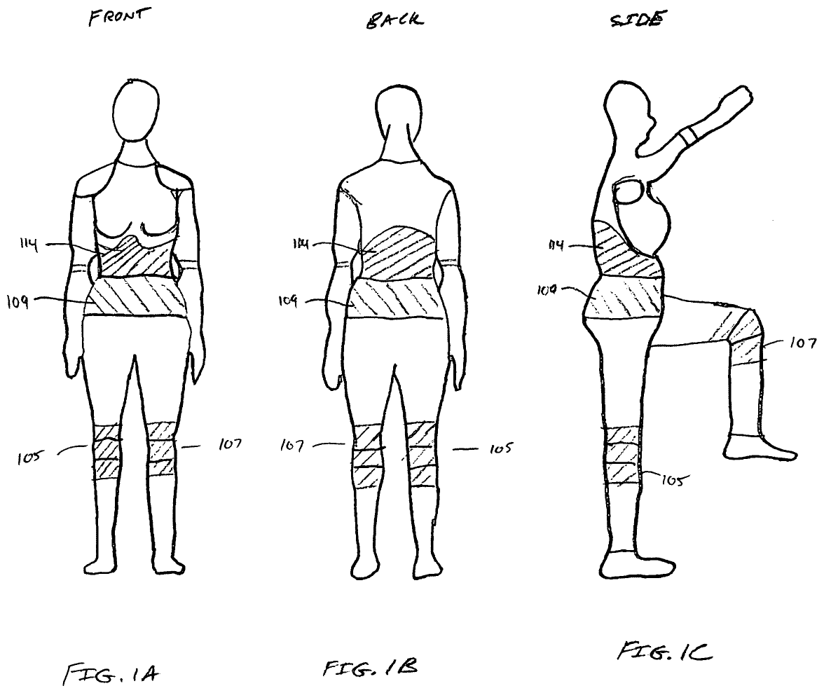

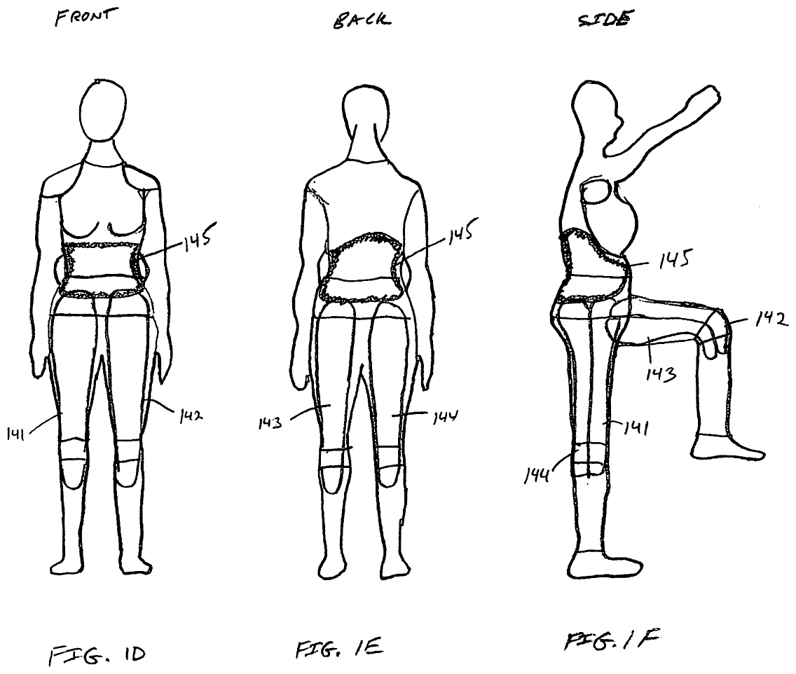

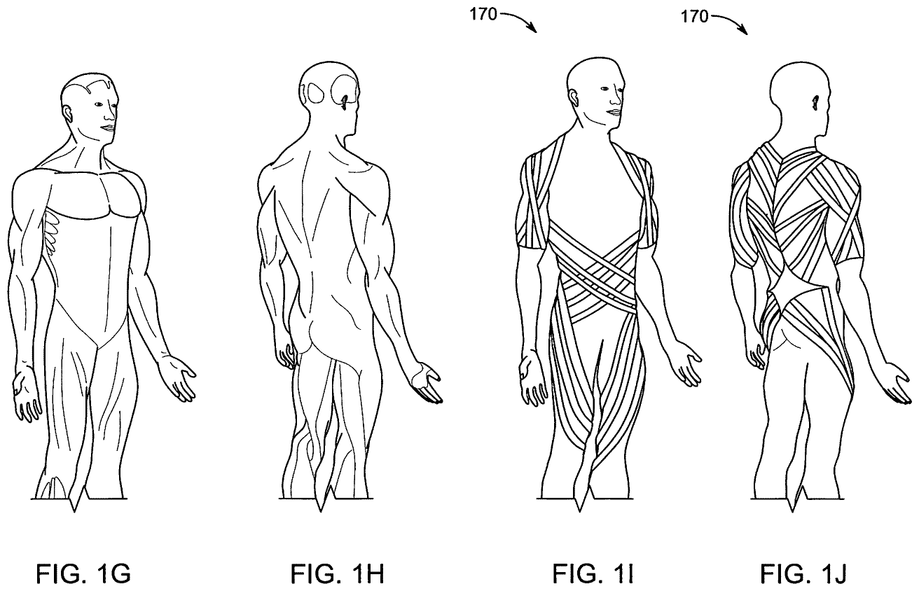

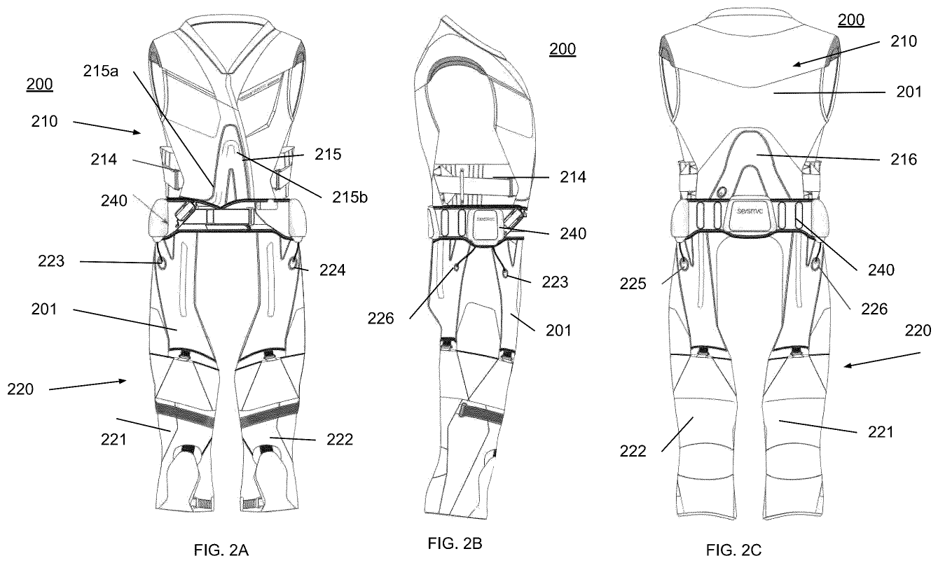

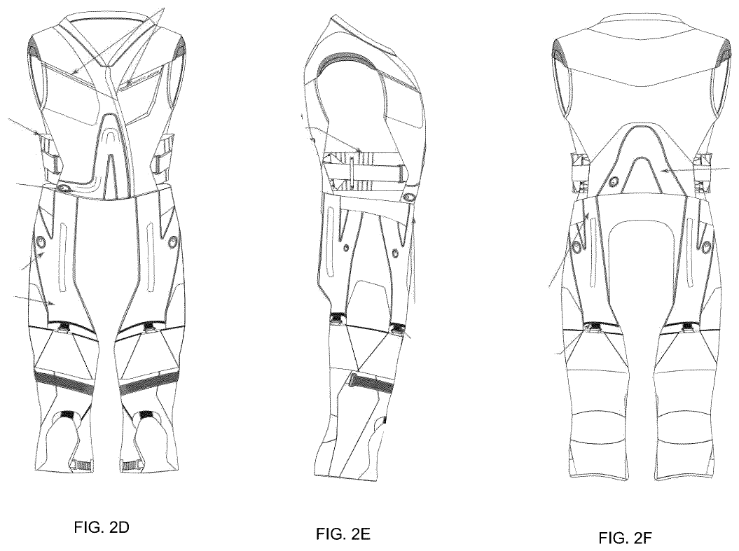

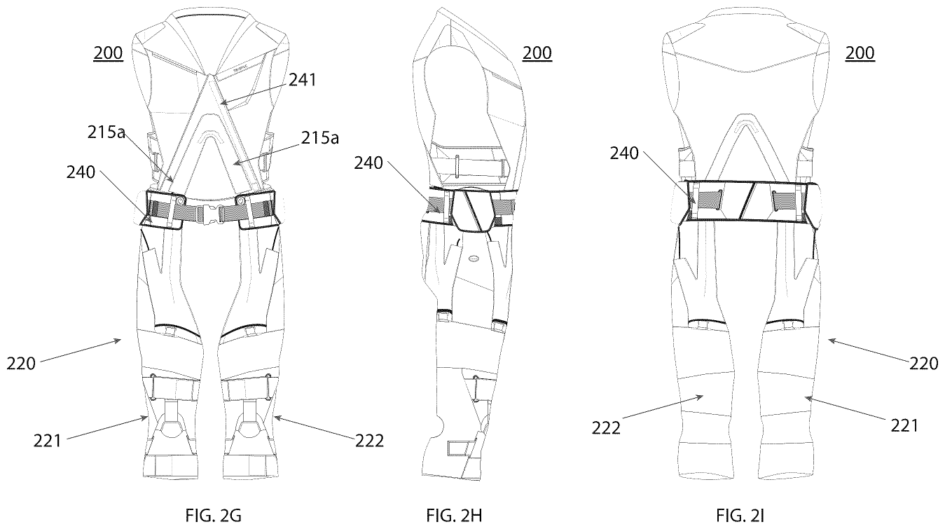

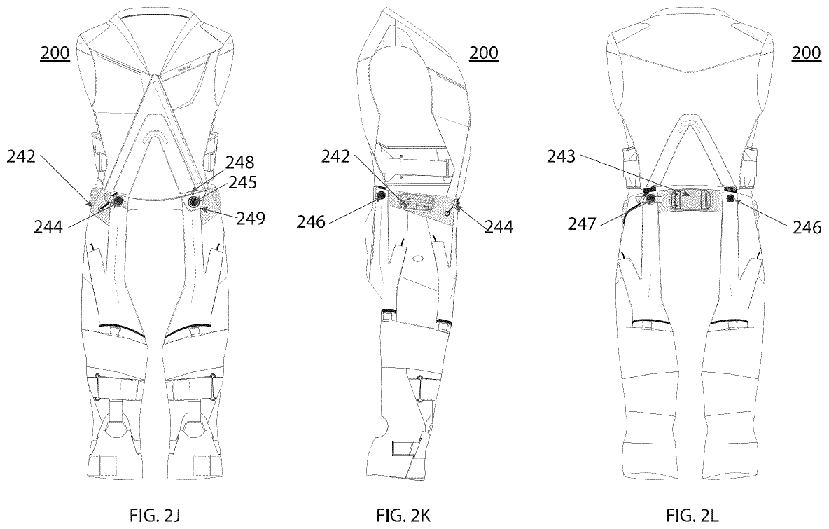

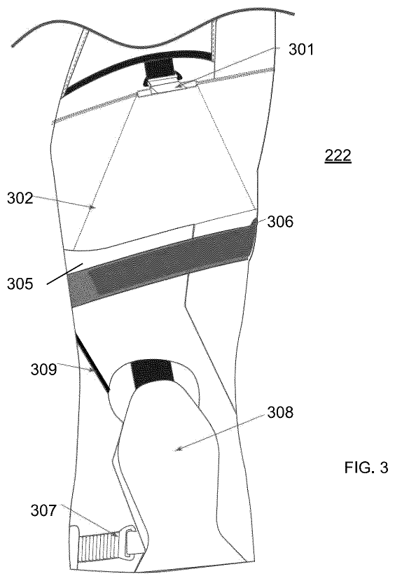

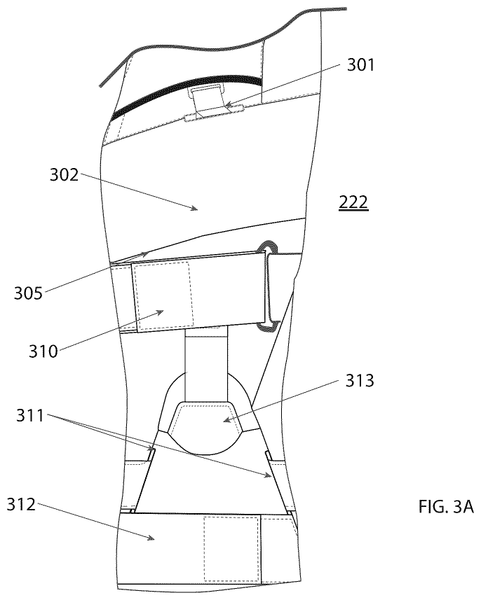

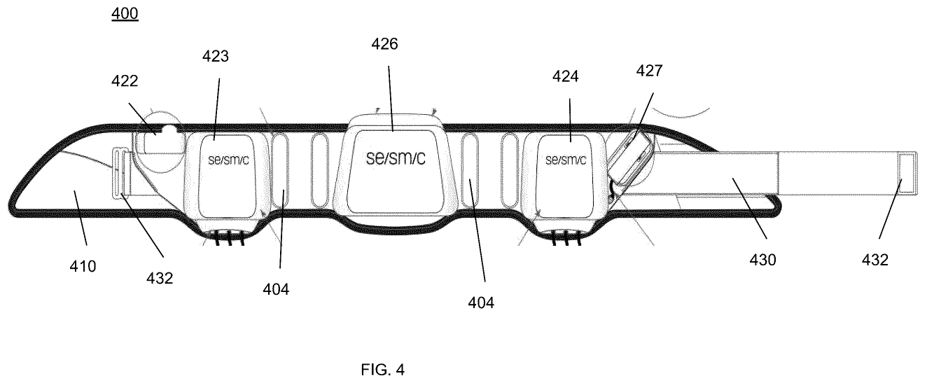

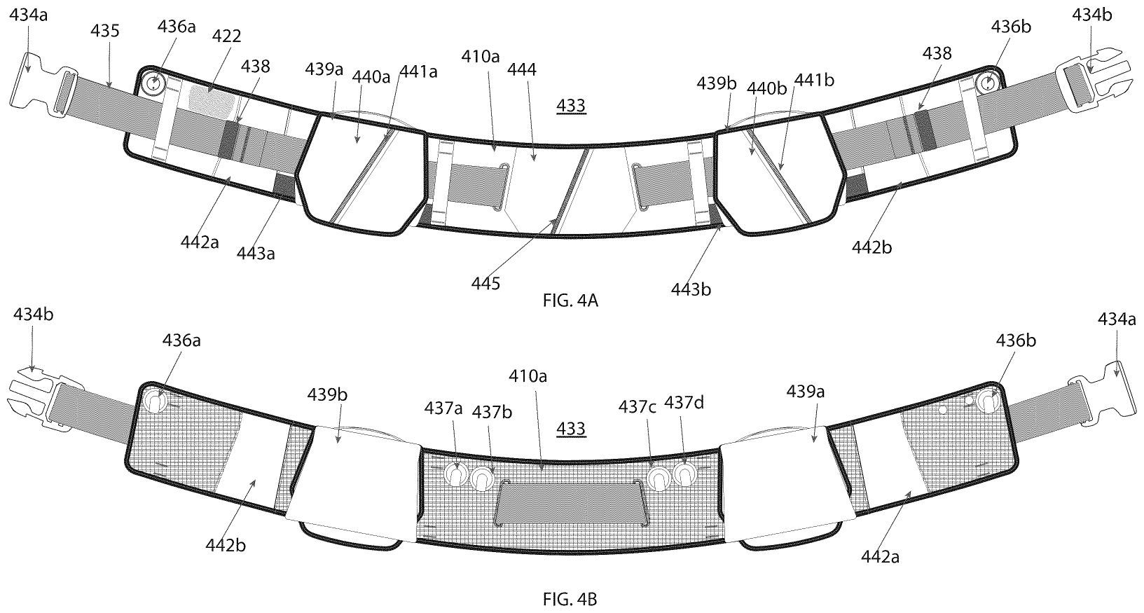

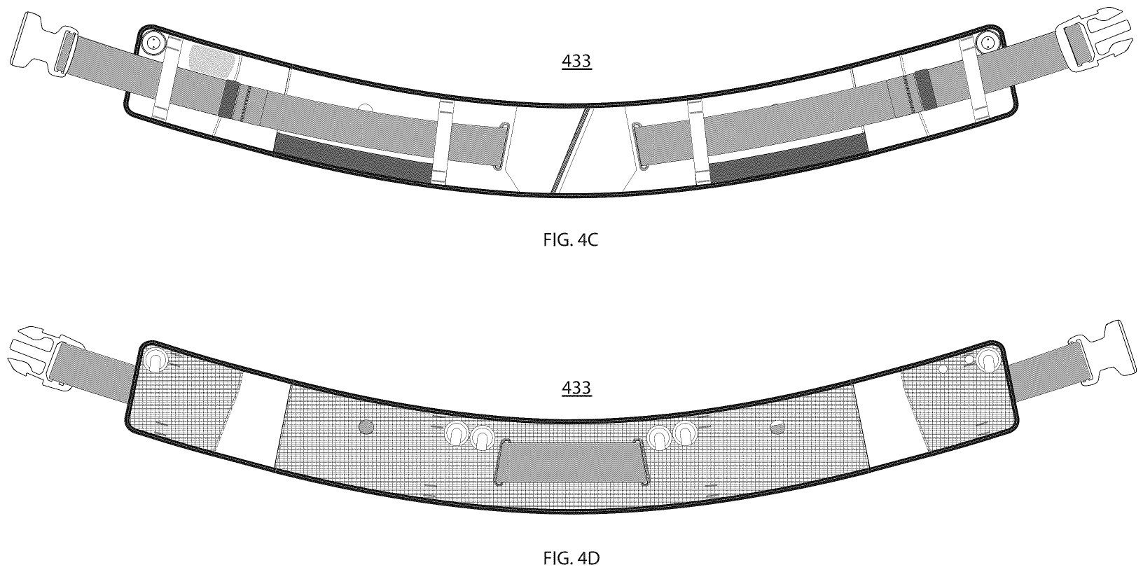

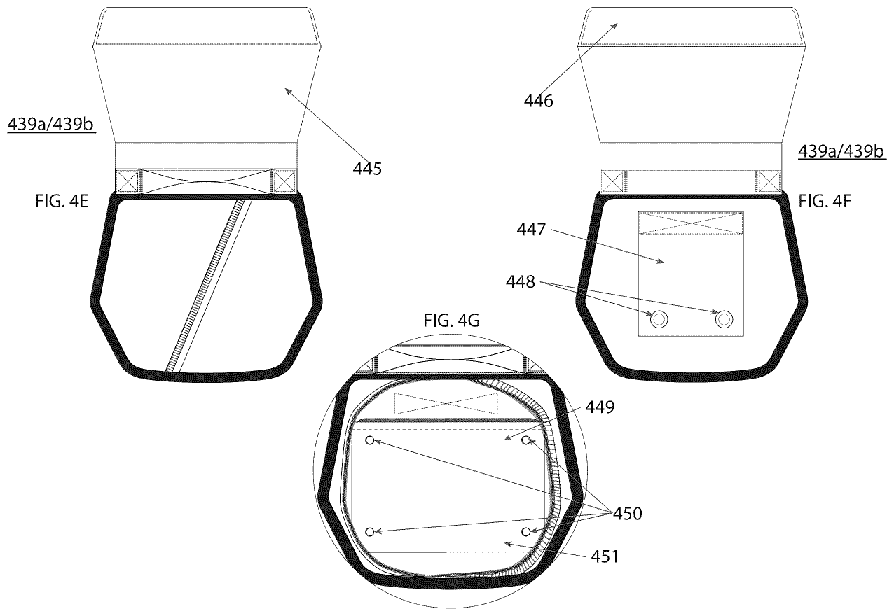

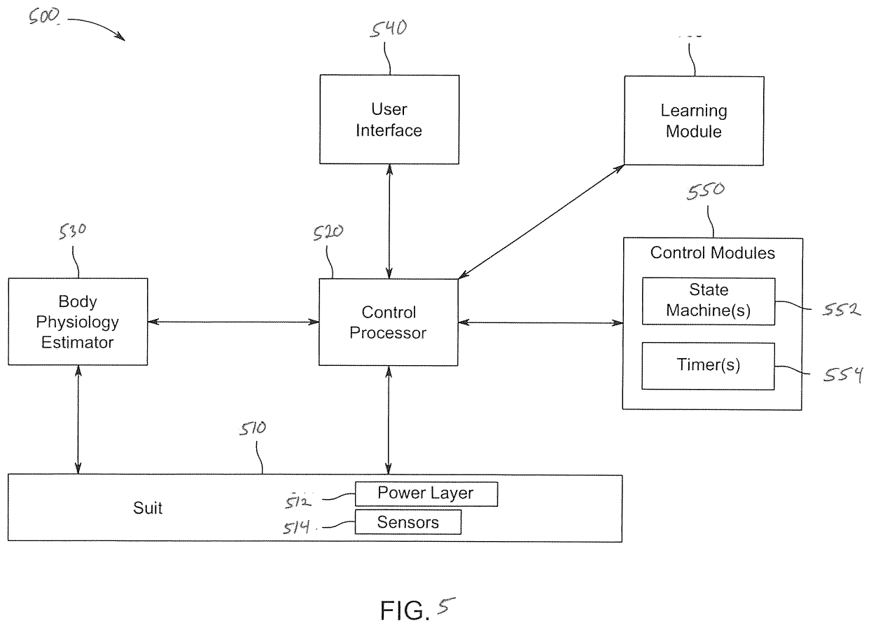

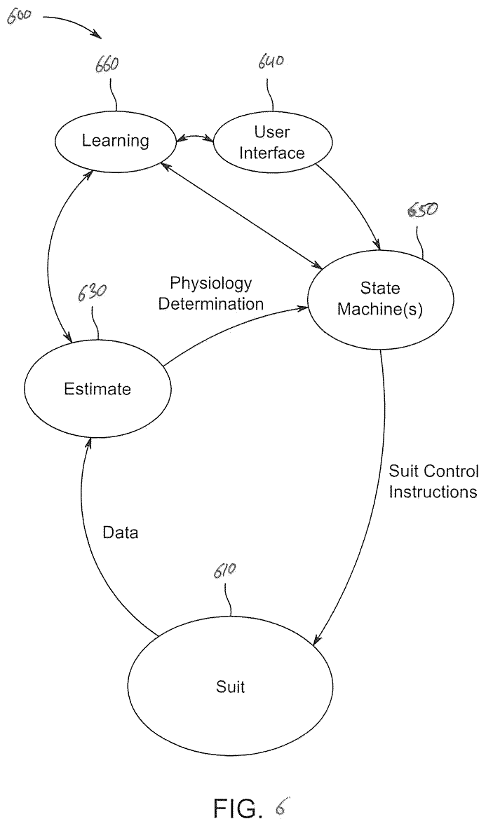

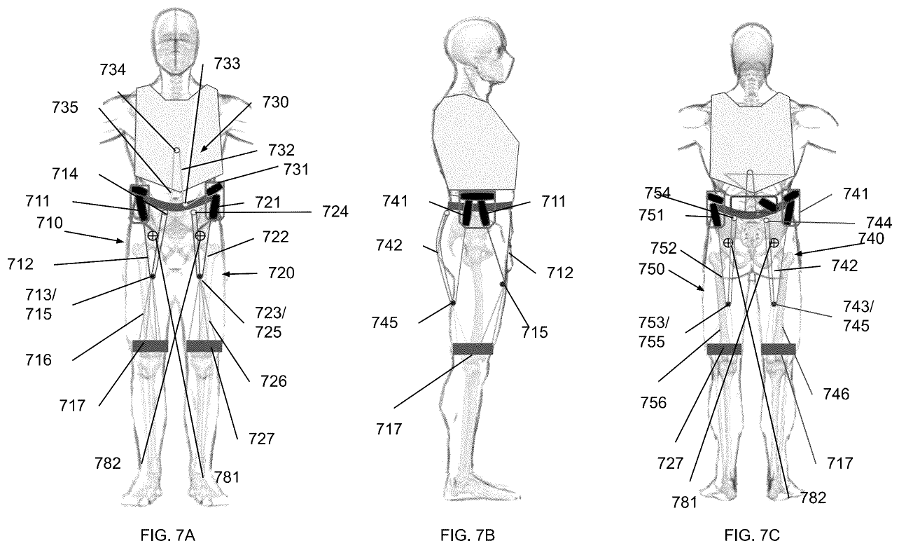

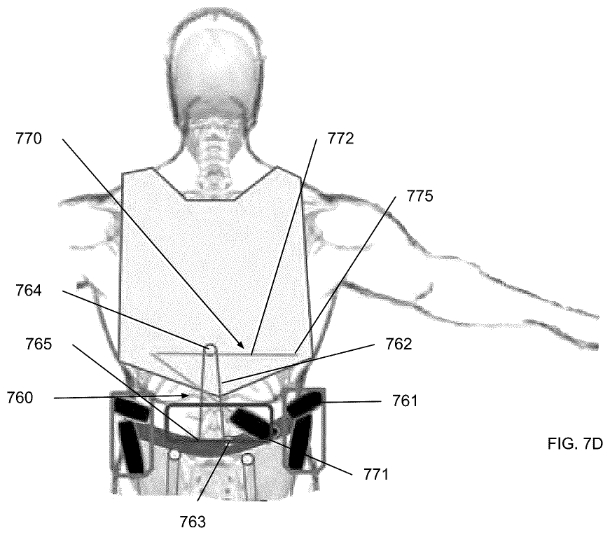

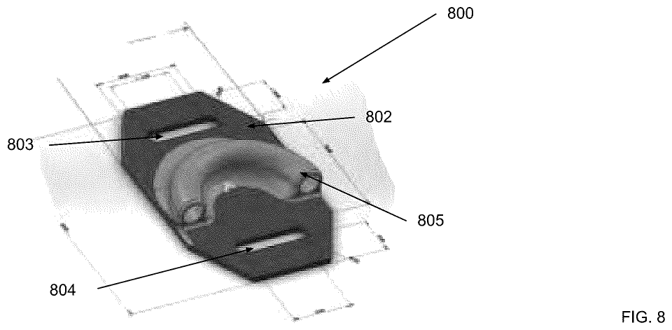

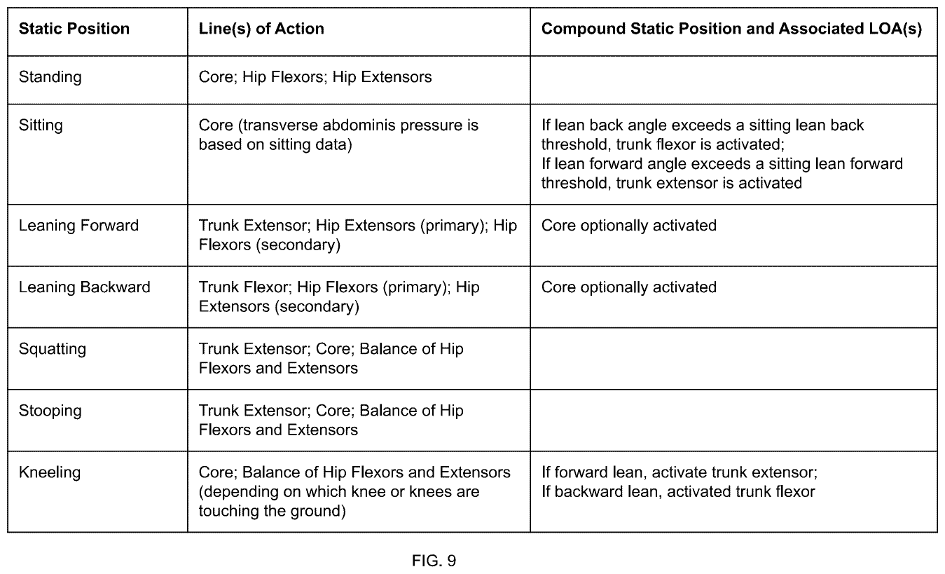

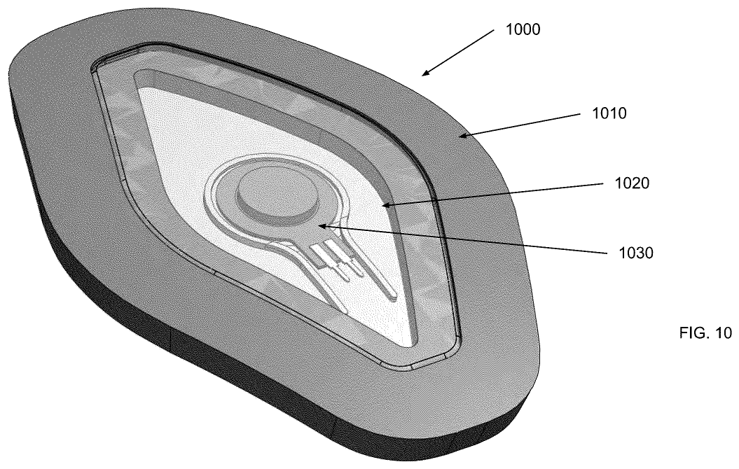

FIGS. 1A-1C show front, back, and side views of a human female, in particular showing potential locations on the body where load distribution members (LDMs) of an oversuit can be positioned according to an embodiment. The LDMs can serve as platforms for the power layers (shown in FIGS. 1D-1F). LDMs can cover or occupy any part of the human body as desired. The LDMs shown in FIGS. 1A-1C are merely illustrative of a few potential locations and it should be appreciated that additional LDMs may be added or certain LDMs may be omitted. The LDMs shown in FIGS. 1A-1C are used primarily to provide flexor and extensor leg muscle assistance and trunk muscle assistance.

Knee LDMs 105 and 107 may be secured around the knee by wrapping around the thigh portion of the leg above the knee and around the calf portion of the leg below the knee. LDMs 105 and 107 are flexible over the knee to allow for knee flex, mobility, comfort, and cooling. A sensor (e.g., a pressure sensor) may be incorporated into LDMs 105 and 107 to sense whether the user is kneeling and resting on one or both knees.

Belt LDM 109 may be secured around the hips and/or waist of the user. Belt LDM 109 may serve as a primary support platform for various subsystem components (e.g., motors, power supply, control unit, etc.) of the oversuit. The primary support platform is designed to handle the weight load and the tensioning load of the subsystems by anchoring the load into the hips, sacrum, and waist of the user (e.g., similar to how a backpackers' hip belt works or that of a soldier's or cop's duty belt).

Core LDM 114 may be secured around the abdomen, sides, and lumbar of the user. Core LDM 114 can exhibit a corset design that is designed to compress around and against the core region of the user.

Although not shown in FIGS. 1A-1C, LDMs may exist for the thighs, shoulders, feet, toes, neck, head, hands, fingers, elbows, or any other suitable body part. Other LDMs and their positions on a human body can be found, for example, in commonly owned U.S. Patent Publication No. 20190283237, which is incorporated by reference herein in its entirety.

As explained above, the LDMs may serve as attachment points for components of the power layer. In particular, the components that provide muscle assistance movements typically need to be secured in at least two locations on the body. This way, when the flexible linear actuators are engaged, the contraction of the actuator can apply a force between the at least two locations on the body. With LDMs strategically placed around the body, the power layer can also be strategically placed thereon to provide any number of muscle assistance movements. For example, the power layer may be distributed across different LDMs or within different regions of the same LDM to approximate any number of different muscles or muscle groups. The power layer may approximate muscle groups such as the abdominals, adductors, dorsal muscles, hip flexors, hip extensors, trunk flexor, trunk extensor, shoulders, arm extensors, wrist extensors, gluteals, arm flexors, wrist flexors, scapulae flexors, thigh flexors, lumbar muscles, foot muscles, pectorals, quadriceps, and trapezii.

FIGS. 1D-1F show front, back, and side views, respectively, of a power layer according to an embodiment. The power layer is shown as multiple segments distributed across and within the various LDMs. As shown, the power layer can include power layer segments 141-145. Each of power layer segments can include any number of flexible linear actuators. Each power layer segment can be secured between two different load distribution members (e.g., between a belt LDM and a knee LDM or a between a belt LDM and a core LDM). Each power layer segment can include a motor and a tensioning element that is connected to the motor. The motor may be connected to a first load distribution member. The tensioning member may have a first end that is connected to the motor and a second end that is connected to a second load distribution member. The tensioning member may be routed through a tube that is integrated within a suit component. In this manner, a power layer segment can exist on the outside of a suit component and within a suit component. In some embodiments, the tube may route the tensioning member through a pully system (that integrated within the suit component) to generated extra power in response to motor action. Some of the power layer segments may exist solely on the anterior side of the body, exist solely on the posterior side, start on the anterior side and wrap around to the posterior side, start on the posterior side and wrap around to the anterior side, or wrap completely around a portion of the body. Power layer segment (PLS) 141 may be secured to LDM 105 and LDM 109, and PLS 142 may be secured to LDM 107 and LDM 109. PLS 143 may be secured to LDM 107 and LDM 108, and PLS 144 may be secured to LDM 105 and LDM 109. PLS 145 may be secured to LDM 109 and LDM 114.

It should be appreciated that the power layer segments are merely illustrative and that additional power layer segments may be added or that some segments may be omitted. In addition, the attachment points for the power layer segments are merely illustrative and that other attachment points may be used.

The human body has many muscles, including large and small muscles that are arranged in all sorts of different configuration. For example, FIGS. 1G and 1H show respective front and back views of a human male's musculature anatomy, which shows many muscles. In particular, the abdominals, adductors, dorsal muscles, shoulders, arm extensors, wrist extensors, gluteals, arm flexors, wrist flexors, scapulae fixers, thigh flexors, lumbar muscles, pectorals, quadriceps, and trapezii are all shown.

The LDMs may be designed so that they can accommodate different sizes of individuals who don the oversuit. For example, the LDMs may be adjusted to achieve the best fit. In addition the LDMs are designed such that the location of the end points and the lines of action are co-located with the bone structure of the user in such a way that the flexdrive placement on the oversuit system are aligned with the actual muscle structure of the wearer for comfort, and the moment arms and forces generated by the flexdrive/oversuit system feel aligned with the forces generated by the wearer's own muscles.

FIGS. 1I and 1J show front and side views of illustrative oversuit 170 having several power layer segments that approximate many of the muscles shown in FIGS. 1G and 1H. The power layer segments are represented by the individual lines that span different parts of the body. These lines may represent specific flexible linear actuators or groups thereof that work together to form the power layer segments that are secured to the LDMs (not shown). As shown, the FLAs may be arrayed to replicate at least a portion of each of the abdominal muscles, dorsal muscles, shoulder muscles, arm extensor and flexor muscles, gluteal muscles, quadriceps muscles, thigh flexor muscles, and trapezii muscles. Thus, oversuit 170 exemplifies one of many possible different power layer segment arrangements that may be used in suits in accordance with embodiments discussed herein. These power layer segments are arranged so that the moment arms and forces generated feel like forces being generated by the user's own muscles, tendons, and skeletal structure. Other possible power layer segment arrangements are illustrated and discussed below.

The power layer segments may be arranged such that they include opposing pairs or groups, similar to the way human muscles are arranged in opposing pairs or groups of muscles. That is, for a particular movement, the opposing pairs or groups can include protagonist and antagonist muscles. While performing the movement, protagonist muscles may perform the work, whereas the antagonist muscles provide stabilization and resistance to the movement. As a specific example, when a user is performing a curl, the biceps muscles may serve as the protagonist muscles and the triceps muscles may serve as the antagonist muscles. In this example, the power layer segments of an oversuit may emulate the biceps and triceps. When the biceps human muscle is pulling to bend the elbow, the oversuit triceps power layer segment can pull on the other side of the joint to resist bending of the elbow by attempting to extend it. The power layer segment can be, for example, either be a FLA operating alone to apply the force and motion, or a FLA in series with an elastic element. In the latter case, the human biceps would be working against the elastic element, with the FLA adjusting the length and thereby the resistive force of the elastic element.

Thus, by arranging the power layer segments in protagonist and antagonist pairs, the power layers segments can mimic or emulate any protagonist and antagonist pairs of the human anatomy musculature system. This can be used to enable oversuits to provide assistive movements, alignment movements, and resistive movements. For example, for any exercise movement requires activation of protagonist muscles, a subset of the power layer segments can emulate activation of antagonist muscles associated with that exercise movement to provide resistance.

The design flexibility of the LDMs and PLSs can enable oversuits to be constructed in accordance with embodiments discussed herein. Using oversuits, the power layer segments can be used to resist motion, assist motion, or align the user's form. In addition, the oversuit can monitor the user's movements and determine whether the user is adhering to “safe” movement practices and can provide notice if the user is not moving in accordance with established movement practices.

FIGS. 2A, 2B, and 2C show front, side, and back views of illustrative oversuit 200 according to an embodiment. Oversuit 200 may embody some or all of the suit components, load distribution member, power layer segments, sensors, a controller, and a user interface, as discussed above. In addition, oversuit 200 may represent one of many different specification implementations of the oversuit shown in FIGS. 1A-1F. Oversuit 200 can include suit component 201 that is constructed to be worn over the user's clothing. Suit component 201 can be a unitary suit that is meant to be worn over the torso, waist, thighs, knees, and calves. Suit component 201 is open in the groin, buttocks, and inner thighs to enable unrestricted toileting and to minimize the amount of material needed to support power layers and load distribution members. In addition, keeping the groin, buttocks, and inner thighs uncovered can prevent unwanted heating. Suit component 201 can be integrated with load distribution members such as knee distribution members 221 and 222 and core distribution member 214 and embedded tunnels for routing flex drive strings from one location to another within the suit. The flex drive strings may remain embedded within suit component 201 and can be attached to and detached from a flexible linear actuator secured to belt 240. Belt 240 may be a separate component from suit component 201 that can be donned and doffed on and off the waist and over suit component 201. Belt 240 may serve as a load distribution member and can support several flexible linear actuators, a battery pack, a controller, and other components. When the user places belt 240 on his or her person, the user may need to connect various belt components to complementary components on suit 201. For example, mechanical connections such as flexible strings and electrical connections such as power and signal routing may need to be connected between belt 240 and suit 201. Conversely, when the user wishes to take belt 240 off, the connections existing between belt 240 and suit 201 are preferably disconnected before belt 240 is removed.

Suit component 201 includes upper body portion 210, which as shown in FIGS. 2A-2C resembles a vest. Upper body portion 210 may include a zipper (not shown), pockets 212, core support member 214, trunk flexor tunnel 215, trunk extensor tunnel 216. The zipper may be closed to close vest-like portion around the torso of the user. In other embodiments, the zipper can be replaced with buttons, hook and loop material, snaps, or other fastening feature. Core support member 214 may be powered by a flexible linear actuator located on belt 240. Core support member 214 may provide transversus abdominis support to the user by compressing core support member 214 around the user. An example of core support member can be found, for example, in commonly owned U.S. Patent Application Publication No. 20190283237, the disclosure of which is incorporated herein in its entirety. Flexor tunnel 215 can exhibit an upside down V shape where the point of the “V” is positioned in line with the sternum of the user. Flexor tunnel 215 can include internal routing features 215a and 215b for redirecting an FLA string (not shown) that is routed through flexor tunnel 215. Internal routing features 215a and 215b can include a curved tube (e.g., see FIG. 8 for example). The FLA string can be connected to a FLA located on belt 240 and to a terminal location within the flexor tunnel. The terminal location can be located near the waist or adjacent to the belly button of the user. When a trunk flexor load is applied by the appropriate FLA, trunk flexion may cause the user's torso to bend forward. Extensor tunnel 216 exhibit an upside down V shape where the point of the “V” is positioned in line with the middle of the user's back. The V shape may be different than the V shape associated with flexor tunnel 215. Extensor tunnel 216 can include internal routing features for redirecting an FLA string (not shown) that is routed through extensor tunnel 215. The FLA string can be connected to a FLA located on belt 240 and to a terminal location within extensor tunnel 216. The terminal location can be located near the waist of the user. When a trunk extensor load is applied by the appropriate FLA, trunk extension may cause the user's torso to bend backward.

Suit component 201 includes lower body portion 220, which extends from the waist down to around the knees. Lower body portion 220 may include load distribution member 221, load distribution member 222, flexor tunnels 223 and 224, and extensor tunnels 225 and 226. FLA strings are routed from FLAs housed on belt 240 through flexor tunnels 223 and 224 and extensor tunnels 225 and 226 to LDMs 221 and 222. Flexor tunnels 223 and 224 and extensor tunnels 225 and 226 can include internal features that re-route the FLA strings internally within suit component 201.

FIGS. 2D-2F show front, side, and back views of illustrative oversuit 200 with belt 240 removed according to an embodiment.

FIGS. 2G, 2H, and 2I show front, side, and back views of an alternative of oversuit 200 according to an embodiment. Oversuit 200 may embody some or all of the suit components, load distribution member, power layer segments, sensors, a controller, and a user interface, as discussed above. In addition to those features previously described for suit 200 in FIGS. 2A, 2B, and 2C, FIG. 2G illustrates a potential variation for front chest flexor tunnel 215a, which in this iteration is attached to tertiary layer 241 including of a multilayer construction and functioning as a zipper cover closure and anchor point for flexdrive string to be housed in 215a (not shown).

Additionally, FIGS. 2J, 2K, and 2L illustrate another embodiment of suit 200 with belt 240 removed so as to show additional potential features Inclusive of integrated lattice padding 242 and 243 (mentioned further below) that can be attached to suit 200 via containment in mesh pocketed compartments. FIGS. 2J, 2K, and 2L also illustrate the inclusion of positive physical connection points 244, 245, 246, and 247 that create a physical lock between suit 200 and belt 240 that not only holds belt 240 in place but can aid in load transfer from suit 200 to belt 240, distributing load to the users hips. Shown in FIGS. 2J, 2K, and 2L is the extension of 241 to incorporate tab 249 which can create both an attachment point for a string anchor points 248, as well as in combination with attachment point 245 help keep flap 241 secure.

FIG. 3 shows a detailed front view of knee load distribution member 222 according to an embodiment. Knee LDM 222 may be constructed primarily from fabric materials with a few hard points or relatively hard points for carrying a load. Knee load distribution member 222 includes attachment point 305, which is connected to plexus 302. Attachment point 301 may be connected to a flexible string (not shown). Plexus 302 may include an array of load lines (not shown) that are connected to loading member 305. Tensioning strap 306 may exist adjacent to and/or overlap loading member 305. Below loading member 305, fabric of LDM 222 may continue down over the knee to tensioning strap 307. Tensing straps 306 and 307 can use any suitable type of tensioning mechanisms such as hook and loop, ratchet buckle, or belt and hole. Knee flap 308 may be a separate component that is integrated into the fabric of LDM 222. Knee flap 308 may include a knee sensor (not shown) that is secured within. Knee flap 308 may be flexible to allow for unrestricted knee bending by the user. Knee flap 308 may include elastic strap that is attached to the top of knee flap 308 to provide free movement for the user. Zipper 309 may be integrated within LDM 222 to facilitate ease of donning and doffing. That is, the user may unzip zipper 309 when donning and doffing LDM 222 and zip zipper 309 while using it.

FIG. 3A shows a detailed front view of another variation of knee load distribution member 222 according to an embodiment. Knee LDM 222 may be constructed primarily from fabric materials with a few hard points or relatively hard points for carrying a load. Knee load distribution member 222 includes attachment point 305, which is connected to plexus 302 (not visible). Attachment point 301 may be connected to a flexible string (not shown) and/or redirect 800 at point 804 (not shown). Plexus 302 may include an array of load lines (not shown) that are connected to loading member 305. Tensioning strap 310 may exist adjacent to and/or overlap loading member 305. Below loading member 305, fabric of LDM 222 may continue down over the knee to tensioning strap 312. Tensing straps 310 and 312 can use any suitable type of tensioning mechanisms such as hook and loop, ratchet buckle, or belt and hole. Knee flap 313 may be a separate component that is integrated into the fabric of LDM 222. Knee sheath 313 may include a knee sensor (not shown) that is secured within. Knee sheath 313 may be flexible to allow for unrestricted knee bending by the user. Knee Sheath 313 may include elastic strap that is attached to the top of knee Sheath 313 and held in place via knee tensioning strap 310 to provide free movement for the user. Slits in the construction of 222 allow for the protrusion and attachment of the arms of sheath 313 to main body of 222 as well as allowance for connection of conductive snaps from sensor (not shown) to main body wiring harness (not shown).

FIG. 4 shows a more detailed view of belt 400 according to an embodiment. Belt 400 may be akin to belt 240. Belt 400 can be multipart construction designed to carry and support the loading actions of the FLAs secured thereto. Belt 400 can be a multi-layer construction with multiple fabric layers, component mounting structures, support stays, power and data routing, padding, and connection mechanism. In one embodiment, belt 400 can include component carrying portion 410 and flexible layer 430. Flexible layer 430 may represent a traditional belt that is at attached to or at least partially sandwiched between layers of component carrying portion 410. Flexible layer 430 may include a fastener 432 such as a Cobra® buckle or a loop back buckle to secure belt 400 to itself and onto the user.

Component carrying portion 410 can include outer layer 401 and an inner layer (not shown). Component mounting structures (not shown), support stays 404, and power and data routing structures (not shown) may be sandwiched between outer layer 401 and the inner layer. Support stays 404 may provide additional structural rigidity to portion 410 (along with rigidity provided by flexible layer 430, component mounting structures and power and data routing structures). Various components may be mounted to component mounting structures on portion 410. These components can include user interface housing 422, hip patch 423, hip patch 424, lumbar patch 426, and other components such as quick connector 427. User interface housing 422 may include an ON/OFF button and LEDs to provide oversuit status. Hip patches 423 and 424 can each include three FLAs and power and data lines that are routed to lumbar patch 426 via power and data routing structures. One FLA can be used for thigh flexor operation, another for thigh extensor operation, and the third can be used for a trunk operation (either flexion or extension). Each of the FLAs in hip patches 423 and 424 may have quick connectors to connected to their respective twisted strings located in the suit component 201. Lumbar patch 426 can include a power source, a controller, a motor for a FLA. The motor may be connected to quick connector 427 via a tunnel (not shown) existing in portion 410. Quick connector 427 may be designed to interface with a twisted string associated with core support member (e.g., member 214).

In some embodiments, hip patch 423, hip patch 424, and lumbar patch 426 can be removed from belt 400 for servicing or replacement. For example, lumbar patch 426 may be swapped out with another lumber patch 426 that has a fully charged power source. As another example, the user may swap out hip patches 423 and 424 with hip patches outfitted with FLAs that have different load capacity ratings. High load capacity FLAs may provide enhanced movement assistance compared to lower load capacity FLAs, but may consume power at a faster rate.

In some embodiments, a lattice structure may be strategically placed at one or more locations on the inner layer side. For example, lattice structures may be placed opposite of hip patches 423 and 424 and lumbar patch 426. Lattice structure may serve multiple purposes: cooling and comfort. Cooling may be achieved because the lattice structure is substantially air permeable, and because the lattice structure this moves belt portion 410 and flexible layer 430 off the body, the wearer can benefit from the cooling effect. Comfort can be achieved because the lattice structure can provide extra padding and forgiveness, yet provide the necessary support for enabling the FLAs to operate and provide the necessary assistive movements. In some embodiments, the lattice structure can be a carbon 3D printed lattice structure.

FIGS. 4A, 4B, 4C and 4D shows detailed views of another possible variation of belt 400 labeled 433 according to an embodiment. Belt 433 may be akin to belt 240. Belt 433 can be multipart construction designed to carry and support the loading actions of the FLAs secured thereto. Belt 433 can be a multi-layer construction with multiple fabric layers, component mounting structures, power and data routing, padding, belt-to-suit connection mechanisms, protective coverings, and closing attachment features. In one embodiment, belt 433 can include component carrying portion 410a and flexible layer 435 with a 2 part side release buckle or similar where 434a represents one side and 434b the opposing side to secure belt 433 to itself and onto the user. Flexible layer 435 may represent a traditional belt that is at attached to or at least partially sandwiched between layers of component carrying portion 410a to secure belt 400 to itself and onto the user, with any excess material from 435 being secured by an elastic loop or similar 438.

Component carrying portion 410a can include an outer layer composed of multiple pieces or panels such as 442a and 442b of different materials performing specific functions for the benefit of the wearer such as stretch and flexibility. In addition, 410a may contain portions of construction such as 444 forming a pocket with an overlapping opening 445 to cover and protect component housings such as previously noted 426. Various components may be mounted to component mounting structures on portion 410a and 435. These components can include user interface housing 422, mechanical attachment points 436a, 436b, and 437a, 437b, 437c, 437d that connect to corresponding points 244, 245, 246, and 247 on suit 200 as well as hook and loop attachments 443a and 433b for securing removable hip pouches 439a and 439b.

FIGS. 4E, and 4F illustrate a potential hip pouch 439a and 439b that in some embodiments hip patch 423, hip patch 424 can be contained within via mechanical attachment or mechanism and covered via textile layer 440a and 440b with an overlap opening such as 441a and 441b to allow access to hip patch 423 and hip patch 424 for removal for service while protecting them and the environment around them. Pouch 439a and pouch 439b could be attached to 435 via flap 447 and secured via snaps 448. this construction cold also include flap 445 that would wrap over and around belt body 410a and attach to hook and loop closure 443a or 443b via opposing closure side 446 on attached to 445. This construction can allow for pouch 439a and 439b to be adjustable for the wearers comfort and be removed from belt 433 for servicing or replacement.

FIG. 4G shows a potential internal construction within pouch 439a and 439b with layer 440a and 440b pulled open via overlap opening 441a and 441b exposing a pocket layer 451 which includes holes 450 for possible screws or bolts for attachment to hip patches 423 and 424 via a mounting plate contained within pocket 451. Label 449 can identify the pouch.

The oversuit may include many sensors in various locations to provide data required by control circuitry to provide such movements. These sensors may be located anywhere on the suit components or the belt. The sensors may provide absolute position data, relative position data, accelerometer data, gyroscopic data, inertial moment data, strain gauge data, resistance data, or any other suitable data. In one embodiment, the oversuit can include four IMUs, and two pressure sensors. The IMUs can provide angle data and speed data. The pressure sensor can detect whether a user is kneeling on the knee containing that sensor. One IMU may be positioned next the right thigh and another IMU may be positioned next to the left thigh. Two additional IMUs may be positioned in line with each other along the back, with the third IMU positioned along the spine near or below the shoulder blades and the fourth IMU may be positioned along the spine near the lumbar region. The data provided by the thigh IMUs may specify the thigh angle and the angular velocity at which the thighs are moving. The data provide by the back IMUs are examined in concert with each other to determine the spine angle. Two IMUs are needed along the back to accurately determine the spine angle because the natural curvature of the spine makes determining the spine angle very difficult with just one IMU on the back.

The oversuit may include a user interface that enables the user to control the oversuit. For example, the user interface can include several buttons or a touch screen interface. The user interface may also include a microphone to receive user spoken commands. The user interface may also include a speaker that can be used to playback voice recordings. Other user interface element such as buzzers (e.g., vibrating elements) may be strategically positioned around the oversuit. In some embodiments, the user interface can be relatively simplistic and include only an ON/OFF switch. In another embodiment, the user interface can include an ON/OFF switch and a positive button (to cause an increase in FLA loading) and a negative button (to cause a decrease in FLA loading). The oversuit may automatically detect the position(s) the user is in and apply static assistance. The user can adjust the level of static assistance provided by the oversuit by pressing the positive and negative buttons.

The oversuit can include communications circuitry such as that contained in lumber patch 416 to communicate directly with a user device (e.g., a smartphone) or with the user device via a central sever. The user may use the user device to select a level of assistance he or she would prefer when the suit is providing static assistance. The oversuit can monitor and record the user's movements and transmit these movements to a central facility (e.g., an insurance company) that can analyze the data to determine whether the user is moving properly or in a manner that is conducive to injury.

The oversuit may be designed to provide static assistance to the user when the user is in a particular position or combination position. For example, the user can benefit from static assistance when he or she is standing, sitting, leaning forward, leaning backward, squatting, stooping, kneeling with a vertical back, kneeling with a forward lean, kneeling with a backward lean. In each of these positions, the oversuit can engage the appropriate power layer segments to assist the user in maintaining position in the relatively static position he or she is in. Static assistance, in contrast to dynamic assistance, aids the user when he or she is relatively stationary and is not actively transitioning from one position to another. Dynamic assistance aids the user in executing transitions from one position to another (e.g., sit to stand or stand to sit).

The oversuit can be operated by electronic controllers disposed on or within the oversuit (e.g., lumbar patch) or in wireless or wired communication with the oversuit. The electronic controllers can be configured in a variety of ways to operate the oversuit and to enable functions of the oversuit. The electronic controllers can access and execute computer-readable programs that are stored in elements of the oversuit or in other systems that are in direct or indirect communications with the oversuit. The computer-readable programs can describe methods for operating the oversuit or can describe other operations relating to a oversuit or to a wearer of an oversuit.

FIG. 5 shows an illustrative symbiosis system 500 according to an embodiment. The symbiosis enables the oversuit to serve as an autonomous overuit nervous system that mimics or emulates the nervous system of a lifeform such as a human being. That is, a nervous system is responsible for basic life functions (e.g., breathing, converting food into energy, and maintaining muscle balance) that are performed automatically without requiring conscious thought or input. The autonomous oversuit nervous system enables the oversuit to automatically provide assistance to the user when and where the user needs it without requiring intervention by the user. System 500 can do this by tracking the user's body physiology and automatically controlling the suit to provide the anticipated or required support and/or assistance. For example, if a user has been standing for a prolonged period of time, one or more of the muscles being used to help the user stand may begin to tire, and a result, the user's body may exhibit signs of fatigue. Suit 500 can observe this muscle fatigue (e.g., due to observed physiological signs) and can automatically cause suit 500 to engage the appropriate power layers to compensate for the muscle fatigue.

Symbiosis of suit 500 may be expressed in different autonomy levels, where each autonomy level represents a degree to which physiological factors are observed and a degree to which suit assistance or movement actions are performed based on the observed physiological factors. For example, the symbiosis levels can range from a zero level of autonomy to absolute full level of autonomy, with one or more intermediate levels of autonomy. As metaphorical example, autonomous cars operate according to different levels, where each level represents a different ability for the car to self-drive. The symbiosis levels of suit operation can be stratified in a similar manner. In a zero level of autonomy, suit 500 may not monitor for any physiological cues, nor automatically engage any suit assistance or movement actions. Thus, in a zero level, the user may be required to provide user input to instruct the suit to perform a desired movement or assistance. In an absolute full level of autonomy, suit 500 may be able to observe and accurately analyze the observed physiological data (e.g., with 99 percent accuracy or more) and automatically execute the suit assistance or movement actions in a way expressly desired by the user. Thus, in the absolute full level, the suit seamlessly serves as an extension of the user's nervous system by automatically determining what the user needs and providing it.

The one or more intermediate levels of autonomy provide different observable physiological results that are accurate but do not represent the absolute nature of the absolute full level of autonomy. For example, the intermediate levels may represent that the suit is fully capable of autonomously performing certain static assistance actions (e.g., support during a forward lean) but not others. A corollary to this is ABS braking; the ABS braking system automatically figures out how best to stop the vehicle without requiring the user to pump the brakes or engage in any other activity other than stepping on the brake pedal. In the suit context, the oversuit knows when the user is leaning forward and engages the appropriate power layer segments to assist the user muscles in maintaining the forward lean. The intermediate levels may also exist while the suit is learning about its user. Each user is different, and the physiological responses are therefore different and particular to each user. Therefore, the ability to discern the physiological cues and the assistance and movements made in response thereto may endure a learning curve before the suit is able to operate at the absolute full level.

FIG. 5 shows that suit system 500 can include suit 510, control processor 520, body physiology estimator 530, user interface 540, control modules 550, and learning module 560. Suit 510 can be any suitable oversuit (e.g., oversuit 200) and can include, among other things, power layer 512 and sensors 514. Control processor 520 may process instructions, pass data, and control the suit. Control processor 520 may be connected to suit 510, body physiology estimator 530, user interface 540, control modules 550, and learning module 560. Control processor 520 may provide signals to suit 510 to control, for example, operation of power layer 512.

Body physiology estimator 530 may receive data inputs from sensors 514, control processor 520, and other components if desired. Estimator 530 is operative to analyze the data to ascertain the physiology of the user. Estimator 530 may apply data analytics and statistics to the data to resolve physiological conditions of the user's body. For example, estimator 530 can determine whether the user is sitting, standing, leaning, laying down, laying down on a side, walking, running, kneeling, jumping, performing exercise movements, playing sports, reaching, holding an object or objects, or performing any other static or active physiological event. The results may be provided to control modules 550, for example, via control processor 520.

Sensors 514 can include an accelerometer, gyroscope, magnetometer, altimeter sensor, EKG sensor, and any other suitable sensor. Sensors 514 may be integrated anywhere within the suit, though certain locations may be more preferred than others. The sensors can be placed near the waist, upper body, shoes, back, thigh, arms, wrists or head. In some embodiments, sensors can be embedded onto the equipment being used by the user. In some embodiments, the sensors can be contained external to the suit. For example, if worn on the wrist or arm of a worker, the device can be embedded into a watch, wrist band, elbow sleeve, or arm band. A second device may be used and clipped on the waist on the pelvis, or slipped into a pocket in the garment, embedded into the garment itself, back-brace, belt, hard hat, protective glasses or other personal protective equipment the worker is wearing. The device can also be an adhesive patch worn on the skin. Other form factors can also clip onto the shoe or embedded into a pair of socks or the shoe itself.

Control modules 550 can include various state machines 552 and timers 554 operative to control operation of suit 510 based on outputs supplied by estimator 530, inputs received via user interface 540, and signals provided by control processor 520. Multiple state machines 552 may control operation of the suit. For example, a master state machine may be supported by multiple slave state machines. The slave state machines may be executed in response to a call from the master state machine. In addition, the slave state machines may execute specific assistance functions or movements. For example, each static assistance operation (e.g., sitting, standing, kneeling, bending forward or backward, squatting, or stooping) or each dynamic assistance operation (sit-to-stand assistance movement, stand-to sit movement, stretch movement, standing movement, walking movement, running movement, jumping movement, crouch movement, specific exercise movement, or any other movement) may have its own slave state machine to control suit operation.

Learning module 560 may be operative to learn preferences, peculiarities, or other unique features of a particular user and feedback the learnings to body physiology estimator 530 and control module 550. In some embodiments, learning module 560 may use data analytics to learn about the user. For example, learning module 560 may learn that a particular user walks with a particular gait and cadence. The gait and cadence learnings can be used to modify state machines 552 that control walking for that user. In another embodiment, learning module 560 may incorporate user feedback received via user interface 540. For example, a user may go through an initial setup process whereby the user is instructed to perform a battery of movements and provide responses thereto so that state machines 552 and timers 554 are set to operate in accordance with the preferences of the user.

FIG. 6 shows illustrative process 600 for implementing symbiosis suit system 500 according to an embodiment. Process 600 includes suit 610, estimator 630, user interface 640, and state machines 650. Process 600 can be represented by a continuous feedback loop in which data is supplied from suit 610 to estimator 630, which provides a physiology determination to state machines 650, which uses the determination to generate suit control instructions that are provided to suit 610. User inputs received via user interface 640 may provide user specified controls that can instruct state machines 650 to execute a particular movement or static assistance. The autonomous suit nervous system is implemented through the continuous feedback loop. The continuous feedback loop enables the autonomous suit nervous system to provide rapid response and control of suit 610. For example, if the user is leaning backwards, the estimator 630 can determine a lean angle as part of the current physiological determination. If the lean angle falls within a predefined range, estimator 630 can confirm that that the user is leaning back and that the appropriate back lean assistance should be provided to assist the user. When the user returns to an upright, non-leaning position, estimator 630 can detect this new position and cease activation of the power layer segments responsible for providing leaning support assistance. Thus, the continuous feedback loop provides real-time assessment and instantaneous suit controls in response to the user's immediate physiological needs, and not after.

In some embodiments, estimator 630 may be able to determine compound positions such as sitting or kneeling in combination with a lean backward or forward. When a compound position is determined, the appropriate state machine 650 may activate the power layer segments to provide the user with assistance to maintain the compound position.

Learning 660 can receive and provide data to estimator 630, user interface 640, and state machines 650. Learning 660 may be leveraged to update state machines 650 and/or estimator 630. If desired, artificial intelligence (AI) may be used by learning 660 to update state machines 650 and/or estimator 630. In other embodiments, state machines 650 may use AI.

FIGS. 7A, 7B, 7C, and 7D show illustrative lines of actions provided by an oversuit according to an embodiment. Each line of action has a start point and an end point. The start point can be an FLA and the end point can be a load distribution member. A line of action may be routed through a twisting string tunnel that is integrated within the suit fabric. Depending on how a line of action is routed from start to end, the line of action can have a 1:1 purchase ratio, 2:1 purchase ratio, 3:1 purchase ratio, or a X:1 purchase ratio, where X is an integer of 2 or more. The purchase ratio can refer to the number of times the twisted string is redirected within the string tunnel. Redirecting the twisted string via string re-route structures within the string tunnel effectively enables the FLA and twisted string to operate according to pulley physics. This can result in increased force generation capacity along the line of action for a unit of action executed by a FLA. In addition, the redirects may speed up tensioning action along the line of action.

FIG. 7A shows lines of actions for the left hip flexor, the right hip flexor, and the trunk flexor. The line of action for right hip flexor 710 can include FLA 711, twisted string 712, re-route structures 713 and 714, string attachment point 715, plexus 716, and knee LDM 717. As shown, twisted string 712 is routed to re-route structure 713, which redirects string 712 to re-reroute structure 714, which re-routes string 712 to string attachment point 715. String attachment point 715 is connected to plexus 716, which distributes load lines from string attachment point 715 to knee LDM 717. During FLA 711 activation, twisted string 712 will pull string attachment point 715 upwards. This upward pull is translated to Knee LDM 717, which is also pulled upwards. The line of action for left hip flexor 720 can include FLA 721, twisted string 722, re-route structures 723 and 724, string attachment point 725, plexus 726, and knee LDM 727. The operation of left hip flexor 720 is similar to that of right hip flexor 710. Both right hip flexor 710 and left hip flexor 720 are shown to have a 3:1 purchase ratio.

The line of action for the trunk flexor can include FLA 731, twisted string 732, re-route structures 733 and 734, and string attachment point 735. Twisted string 732 is routed to re-route structure 733, which redirects string 732 to re-reroute structure 734, which redirects string 732 to string attachment point 735. During FLA 731 activation, twisted string 732 will pull the user's chest forward (i.e., into a forward lean) at re-route structure 734. The trunk flexor is shown to have a 2:1 purchase ratio.

FIG. 7C shows lines of actions for left hip extensor, right hip extensor, trunk extensor, and core support. The line of action for right hip extensor 740 can include FLA 741, twisted string 742, re-route structures 743 and 744, string attachment point 745, plexus 746, and knee LDM 747. As shown, twisted string 742 is routed to re-route structure 743, which redirects string 742 to re-reroute structure 744, which re-routes string 742 to string attachment point 745. String attachment point 745 is connected to plexus 746, which distributes load lines from string attachment point 745 to knee LDM 747. During FLA 741 activation, twisted string 742 will pull string attachment point 745 upwards. This upward pull is translated to Knee LDM 717, which is also pulled upwards. The line of action for left hip extensor 750 can include FLA 751, twisted string 752, re-route structures 753 and 754, string attachment point 755, plexus 756, and knee LDM 727. The operation of left hip extensor 750 is similar to that of right hip flexor 710. Both right hip extensor 740 and left hip extensor 750 are shown to have a 3:1 purchase ratio.

FIG. 7D shows the line of action for the trunk extensor can include FLA 761, twisted string 762, re-route structures 763 and 764, and string attachment point 765. Twisted string 762 is routed to re-route structure 763, which redirects string 762 to re-reroute structure 764, which redirects string 762 to string attachment point 765. During FLA 761 activation, twisted string 762 will pull the user's back backward (i.e., into a backward lean) at re-route structure 764. The trunk extensor is shown to have a 2:1 purchase ratio.

The line of action for core support 770 can include FLA 771, twisted string 772, and string attachment point 775. Twisted string 772 may be routed to a core support member (not shown) that is designed to provide compressive force to the transverse abdominis. When FLA 771 is activated, compressive force is provided to the transverse abdominis via the core support member. The core support line of action is shown to have a 1:1 purchase ratio.

FIG. 7B shows a side view of right hip flexor 710 and right hip extensor 740. Note that the string attachment point 715 is positioned higher up on the leg than string attachment point 745. Further note that re-route structures 714 and 744 are substantially coplanar to each other. FIGS. 7A and 7B show left and right hip center points 781 and 782. Center points 781 and 782 may serve as reference points for where re-reroute structure and string attachment point are positioned within the oversuit. For example, re-reroute structures 714 and 724 and re-route structures 744 and 754 may be positioned above and inside respective center points 781 and 782. Attachment points 715, 725, 745, and 755 may be positioned below but in line with respective center points 781 and 782.

FLAs 711, 741, and 761 may be contained in a right thigh patch that is mounted to a belt support member. FLAs 721, 751, and 731 may be contained in a left thigh patch that is mounted to the belt support member. FLA 771 may be contained in a lumbar patch that is mounted to the belt support member. Twisted strings 712, 722, 732, 742, 752, and 762 may be routed through twisted string tunnels that are integrated within the fabric of the suit (such the strings are not exposed). The re-route structures may also be integrated within the fabric such that the structures are not exposed.

It should be understood that the lines of actions shown in FIGS. 7A-7C are merely illustrative and that additional arrangements may be possible.

FIG. 8 shows illustrative re-route structure 800 according to an embodiment. Structure 800 can include substrate 802 with optional integrated slots 803 and 804. Webbing, straps, or tubular nylon can be inserted through slots 803 and 804. Curved tube member 805 is mounted to substrate 802 and includes a hollow tube through which a twisted string can be routed. The inside diameter of the hollow tube may be sized to provide ample space for the twisted string to unravel or expand when not in use. In addition, the radius of curved tube member 805 may be selected to prevent binding or crimping of the string when is being twisted by an FLA.

FIG. 9 shows an illustrative chart of which lines of action are used for a particular static position according to an embodiment. In addition, the chart in FIG. 9 also shows which lines of action are used for compound static positions. In the sitting position, the core, hip flexors, and hip extensors may be used to provide support to the user when he or she is standing in a relatively static position. In the sitting position, the core may be optionally used. In some embodiments, the core support may be activated such that a minimal transverse abdominis pressure is applied or the pressure is proportional to sitting data being received by the sensors. If, while the user is sitting, the user leans back to an extent that a sitting lean back threshold is exceeded, the trunk flexor can be activated to support the backward lean. If, while the user is sitting, the user leans forward to an extent that a sitting lean forward threshold is exceeded, the trunk extensor can be activated to support the forward lean.

In the leaning forward static position, the trunk extensor is activated. The hip flexors and hip extensors may also be activated. The hip extensors may serve as the primary hip line of action and the hip flexors may serve as the second hip line of action. In some embodiments, the hip flexors and hip extensors are both activated, but the hip extensors are carrying more load than the hip flexors. In another embodiment, only the trunk extensor and hip extensors are activated. The core can be optionally activated.

In the leaning backward static position, the trunk flexor is activated. The hip flexors and hip extensors may also be activated. The hip flexors may serve as the primary hip line of action and the hip extensors may serve as the second hip line of action. In some embodiments, the hip flexors and hip extensors are both activated, but the hip flexors are carrying more load than the hip extensors. In another embodiment, only the trunk flexor and hip flexors are activated. The core can be optionally activated.

In the squatting static position, the trunk extensor, core, and a balancing of the hip flexors and hip extensors can be activated. The balancing may be based on the squat position of the user.

In the stooping static position, the trunk extensor, core, and a balancing of the hip flexors and hip extensors can be activated. The balancing may be based on the stooped position of the user.

In the kneeling position, the core and a balancing of the hip flexors and hip extensors can be activated. The left hip flexors and extensors may be active when the left knee sensor indicate that the left knee is on the ground. The right hip flexors and extensors may be active when the right knee sensor indicate that the right knee is on the ground. If the user leans forward while kneeling, the truck extensor may be activated. If the user leans backward while kneeling, the truck flexor may be activated.

In some scenarios, there is a need to distinguish between kneeling and squatting postures. Both postures have the thighs tilted somewhere in the vicinity of 90° to the vertical. Kneeling means one or both knees are touching the ground, whereas in squatting neither knee touches the ground. These two postures are sufficiently similar that using the lower back IMU and the two thigh patch IMUs does not provide sufficient discriminating power between them. The thigh pitch angles of the two postures are too similar. To address this issue, the oversuit incorporates additional sensors, one on each knee, that are designed to activate when the suit wearer puts a significant amount of pressure on one or both knee sensors. The knee sensor data can be used as an additional condition to distinguish kneeling from squatting.

A variety of knee sensors can be used. One example includes resistive pressure sensors where the resistance value changes when pressure is applied to the device and mechanical switches (such as metallic dome switches which have a positive depressive action, aka a “click”, when the applied force reaches a trigger point).

The pressure sensor can be located on the front of the knee just below the kneecap. Other possible designs include using multiple pressure sensors located above, below, or to the side of the front of the kneel sensor in various arrangements. These additional sensors can increase the likelihood of proper kneeling detection if the user is in an awkward position or encumbered by nearby objects. All knee sensors can be incorporated into the knee fabric cover the knee.

To reduce the likelihood of creating any user experience issues for the suit wearer, such as feeling any lumpiness or discomfort when kneeling, resistive pressure sensors such as the FSR UX Force Sensing Resistors® by Interlink Electronics can be used. These sensors have a total thickness as small as less than ½ mm and are suitable for embedding into the garment fabric. FIG. 10 show a knee sensor enclosure 1000 that can be embedded into the garment fabric. Knee sensor enclosure 1000 can place the pressure sensor inside a soft, padded enclosure that is thin and flexible enough to wrap around the front of the knee just below the kneecap that can be embedded into the garment. Knee sensor enclosure 1000 can include first substrate 1010 that forms an outer layer, second substrate 1020, which is contained within the boundaries of first substrate 1010 and encapsulates pressure switch 1030.

Electrical connections to the knee sensors are required to transmit the sensor readings to the suit electronics. Depending on sensor type, the data cables will be either a single twisted pair or two twisted pairs per sensor. Onboard logic in the suit electronics will read out the sensor values and pass on the information to the activity class estimator and the firmware's control logic. For a resistive pressure sensor, a typical response curve of resistance vs force can govern operating characteristics of the sensor. Assuming that a suit wearer with a minimum weight of approximately 100 pounds puts half of his/her weight on each leg, and assuming that each leg's portion is distributed equally between the knee and the foot when the person is in a kneeling position, then the minimum amount of weight that should be certain to trigger the knee sensor would be about 25 pounds (11 kg/11000 g). In one embodiment, the minimum weight for triggering the sensor can be set a bit below this at around 15 pounds (7 kg/7000 g). Such a setting has been found to prevent most accidental activations and false positives while allowing true kneeling events to be reliably detected. To avoid accidental knee sensor triggers due to the wearer pressing a knee sensor against some object when not in a kneeling posture, a timing threshold can be imposed. That is a minimum timing threshold may be imposed on the knee sensor detection to prevent false positives. The minimum timing threshold can be a few to ten seconds, to reduce the likelihood that brief, accidental bumps against the sensor would cause a trigger. For two-knee kneeling, timing thresholds can also be imposed to prevent false positives. Other conditions can be imposed. For example, the measured pitch angles of the two thigh sensors may require that they be oriented in a manner consistent with kneeling. When all these requirements are satisfied simultaneously, kneeling can be detected with a high degree of efficiency and a very low false positive rate.

The knee sensor, its enclosure, and accompanying electrical connections are able to withstand a minimum number of washing and drying cycles in the industrial laundry environment including any harsh chemicals used in the laundering process. The sensor and its connections to the data cables can be potted or otherwise water-proofed and housed in pockets built into the suit lining or attached between layers of the garment. These components are also designed to withstand the normal wear and tear of day-to-day use in the industrial environment.

FIGS. 11A-11G show different views of oversuit 1100 according to an embodiment. Oversuit 1100 includes fabric suit component 1110 that is worn by the user and belt member 1150 that is worn on top of fabric suit component 1110. Fabric suit component 1110 covers the upper body and a portion of the lower body, as shown. Fabric suit component 1110 includes twisted string tunnels that are embedded in the fabric of the suit, core support member 1120, and knee LDMs 1131 and 1132. FIGS. 11C and 11D show trunk flexor string tunnel 1112. FIGS. 11E and 11F show trunk extensor tunnel 1114. Belt member 1150 includes thigh patch 1151, thigh patch 1152, and lumbar patch 1153, as shown. No FLAs or their respective strings are shown.

FIGS. 12A-12D show another example of a knee sensor design and construction 1210 for a suit that uses basic garment construction techniques to create a soft, wearable resistor that sends a signal to the suit when pressure is applied to the sensor. This causes the soft sensor to reach a predetermined resistance (measure in ohms) threshold in the circuit indicating that the wearer is in a kneeling position with weight on their knees.

The Sensor assembly 1210 can include five main layers of material: an outer layer on each side of a polyester spacer mesh 1217 and 1218 that provides minimal cushion and primarily holds the other material together. Attached at the inside of each piece of spacer mesh is a cut piece of conductive, copper infused woven material 1211 and 1212. Each piece of conductive material has a lead extending out to the side that serves as a connection point to the wiring harness. This lead is backed with an interfacing 1213 and 1214 to stabilize it, and then a conductive metal snap 1216 is attached at the same time sandwiching a wire lead 1215 Shown in FIG. 12A. The conductive materials can be attached directly separately via standard lock-stitch to each individual spacer mesh piece 1217 and 1218 shown in FIG. 12B. These assemblies then sandwich a resistive material (Velostat 6mil, or Eeotex or similar) 1219 as shown in FIG. 12C and are sewn together in that configuration with an edge lock-stitch to complete assembly 1210.

FIGS. 13A-13D Show one possible method of containment and attachment for a soft knee sensor assembly such as 1210 in which a protective sleeve or sheath 1310 is created using standard garment and softgoods sewing construction techniques and materials and attached to suit 200 at lower leg 221 and 222. Of note Sheath 1310 may be analogous to sheath 313 from FIG. 3A.

FIG. 13A shows the outside facing view of an example of such a sheath 1310, where outer textile layer 1322 is sewn clean finished to body side facing layer 1323, where 1322 is a durable textile and 1323 is a softer textile. In the process of attaching these materials an elastic band 1321 with velcro loop 1320 attached is caught in at the top edge for attachment to the garment leg 221/222. FIG. 13B illustrates the body facing view of 1310 in which a foam pad 1324 could be included for wearers comfort. Also shown are attachment hook and loop pieces 1325 that secure 1310 at either side to outer shell of leg 221/222, and also have holes cut in them for access to conductive snap connections via 1216 to the main garment wiring harness.

FIGS. 13C and 13D illustrate how sheath 1310 can house sensor 1210 and attach to garment 200 at lower leg 221/222.

FIG. 14 illustrates an example oversuit 1800 that includes actuators 1801, sensors 1803, and a controller configured to operate elements of oversuit 1800 (e.g., 1801, 1803) to enable functions of the oversuit 1800. The controller 1805 is configured to communicate wirelessly with a user interface 1810. The user interface 1810 is configured to present information to a user (e.g., a wearer of the oversuit 1800) and to the controller 1805 of the flexible oversuit or to other systems. The user interface 1810 can be involved in controlling and/or accessing information from elements of the oversuit 1800. For example, an application being executed by the user interface 1810 can access data from the sensors 1803, calculate an operation (e.g., to apply dorsiflexion stretch) of the actuators 1801, and transmit the calculated operation to the oversuit 1800. The user interface 1810 can additionally be configured to enable other functions; for example, the user interface 1810 can be configured to be used as a cellular telephone, a portable computer, an entertainment device, or to operate according to other applications.

The user interface 1810 can be configured to be removably mounted to the oversuit 1800 (e.g., by straps, magnets, Velcro, charging and/or data cables). Alternatively, the user interface 1810 can be configured as a part of the oversuit 1800 and not to be removed during normal operation. In some examples, a user interface can be incorporated as part of the oversuit 1800 (e.g., a touchscreen integrated into a sleeve of the oversuit 1800) and can be used to control and/or access information about the oversuit 1800 in addition to using the user interface 1810 to control and/or access information about the oversuit 1800. In some examples, the controller 1805 or other elements of the oversuit 1800 are configured to enable wireless or wired communication according to a standard protocol (e.g., Bluetooth, ZigBee, WiFi, LTE or other cellular standards, IRdA, Ethernet) such that a variety of systems and devices can be made to operate as the user interface 1810 when configured with complementary communications elements and computer-readable programs to enable such functionality.

The oversuit 1800 can be configured as described in example embodiments herein or in other ways according to an application. The oversuit 1800 can be operated to enable a variety of applications. The oversuit 1800 can be operated to enhance the strength of a wearer by detecting motions of the wearer (e.g., using sensors 1803) and responsively applying torques and/or forces to the body of the wearer (e.g., using actuators 1801) to increase the forces the wearer is able to apply to his/her body and/or environment. The oversuit 1800 can be operated to train a wearer to perform certain physical activities. For example, the oversuit 1800 can be operated to enable rehabilitative therapy of a wearer. The oversuit 1800 can operate to amplify motions and/or forces produced by a wearer undergoing therapy in order to enable the wearer to successfully complete a program of rehabilitative therapy. Additionally or alternatively, the oversuit 1800 can be operated to prohibit disordered movements of the wearer and/or to use the actuators 1801 and/or other elements (e.g., haptic feedback elements) to indicate to the wearer a motion or action to perform and/or motions or actions that should not be performed or that should be terminated. Similarly, other programs of physical training (e.g., dancing, skating, other athletic activities, vocational training) can be enabled by operation of the oversuit 1800 to detect motions, torques, or forces generated by a wearer and/or to apply forces, torques, or other haptic feedback to the wearer. Other applications of the oversuit 1800 and/or user interface 1810 are anticipated.

The user interface 1810 can additionally communicate with communications network(s) 1820. For example, the user interface 1810 can include a WiFi radio, an LTE transceiver or other cellular communications equipment, a wired modem, or some other elements to enable the user interface 1810 and oversuit 1800 to communicate with the Internet. The user interface 1810 can communicate through the communications network 1820 with a server 1830. Communication with the server 1830 can enable functions of the user interface 1810 and oversuit 1800. In some examples, the user interface 1810 can upload telemetry data (e.g., location, configuration of elements 1801, 1803 of the oversuit 1800, physiological data about a wearer of the oversuit 1800) to the server 1830.

In some examples, the server 1830 can be configured to control and/or access information from elements of the oversuit 1800 (e.g., 1801, 1803) to enable some application of the oversuit 1800. For example, the server 1830 can operate elements of the oversuit 1800 to move a wearer out of a dangerous situation if the wearer was injured, unconscious, or otherwise unable to move themselves and/or operate the oversuit 1800 and user interface 1810 to move themselves out of the dangerous situation. Other applications of a server in communications with a oversuit are anticipated.

The user interface 1810 can be configured to communicate with a second user interface 1845 in communication with and configured to operate a second flexible oversuit 1840. Such communication can be direct (e.g., using radio transceivers or other elements to transmit and receive information over a direct wireless or wired link between the user interface 1810 and the second user interface 1845). Additionally or alternatively, communication between the user interface 1810 and the second user interface 1845 can be facilitated by communications network(s) 1820 and/or a server 1830 configured to communicate with the user interface 1810 and the second user interface 1845 through the communications network(s) 1820.

Communication between the user interface 1810 and the second user interface 1845 can enable applications of the oversuit 1800 and second oversuit 1840. In some examples, actions of the oversuit 1800 and second flexible oversuit 1840 and/or of wearers of the oversuit 1800 and second oversuit 1840 can be coordinated. For example, the oversuit 1800 and second oversuit 1840 can be operated to coordinate the lifting of a heavy object by the wearers. The timing of the lift, and the degree of support provided by each of the wearers and/or the oversuit 1800 and second oversuit 1840 can be controlled to increase the stability with which the heavy object was carried, to reduce the risk of injury of the wearers, or according to some other consideration. Coordination of actions of the oversuit 1800 and second oversuit 1840 and/or of wearers thereof can include applying coordinated (in time, amplitude, or other properties) forces and/or torques to the wearers and/or elements of the environment of the wearers and/or applying haptic feedback (though actuators of the oversuits 1800, 1840, through dedicated haptic feedback elements, or through other methods) to the wearers to guide the wearers toward acting in a coordinated manner.

The oversuit 1800 can be operated to transmit and/or record information about the actions of a wearer, the environment of the wearer, or other information about a wearer of the oversuit 1800. In some examples, kinematics related to motions and actions of the wearer can be recorded and/or sent to the server 1830. These data can be collected for medical, scientific, entertainment, social media, or other applications. The data can be used to operate a system. For example, the oversuit 1800 can be configured to transmit motions, forces, and/or torques generated by a user to a robotic system (e.g., a robotic arm, leg, torso, humanoid body, or some other robotic system) and the robotic system can be configured to mimic the activity of the wearer and/or to map the activity of the wearer into motions, forces, or torques of elements of the robotic system. In another example, the data can be used to operate a virtual avatar of the wearer, such that the motions of the avatar mirrored or were somehow related to the motions of the wearer. The virtual avatar can be instantiated in a virtual environment, presented to an individual or system with which the wearer is communicating, or configured and operated according to some other application.

Conversely, the oversuit 1800 can be operated to present haptic or other data to the wearer. In some examples, the actuators 1801 (e.g., twisted string actuators, exotendons) and/or haptic feedback elements (e.g., EPAM haptic elements) can be operated to apply and/or modulate forces applied to the body of the wearer to indicate mechanical or other information to the wearer. For example, the activation in a certain pattern of a haptic element of the oversuit 1800 disposed in a certain location of the oversuit 1800 can indicate that the wearer had received a call, email, or other communications. In another example, a robotic system can be operated using motions, forces, and/or torques generated by the wearer and transmitted to the robotic system by the oversuit 1800. Forces, moments, and other aspects of the environment and operation of the robotic system can be transmitted to the oversuit 1800 and presented (using actuators 1801 or other haptic feedback elements) to the wearer to enable the wearer to experience force-feedback or other haptic sensations related to the wearer's operation of the robotic system. In another example, haptic data presented to a wearer can be generated by a virtual environment, e.g., an environment containing an avatar of the wearer that is being operated based on motions or other data related to the wearer that is being detected by the oversuit 1800.

Note that the oversuit 1800 illustrated in FIG. 14 is only one example of a oversuit that can be operated by control electronics, software, or algorithms described herein. Control electronics, software, or algorithms as described herein can be configured to control flexible oversuits or other mechatronic and/or robotic system having more, fewer, or different actuators, sensors or other elements. Further, control electronics, software, or algorithms as described herein can be configured to control oversuits configured similarly to or differently from the illustrated oversuit 1800. Further, control electronics, software, or algorithms as described herein can be configured to control flexible oversuits having reconfigurable hardware (i.e., oversuits that are able to have actuators, sensors, or other elements added or removed) and/or to detect a current hardware configuration of the flexible oversuits using a variety of methods.

A controller of a oversuit and/or computer-readable programs executed by the controller can be configured to provide encapsulation of functions and/or components of the flexible oversuit. That is, some elements of the controller (e.g., subroutines, drivers, services, daemons, functions) can be configured to operate specific elements of the oversuit (e.g., a twisted string actuator, a haptic feedback element) and to allow other elements of the controller (e.g., other programs) to operate the specific elements and/or to provide abstracted access to the specific elements (e.g., to translate a command to orient an actuator in a commanded direction into a set of commands sufficient to orient the actuator in the commanded direction). This encapsulation can allow a variety of services, drivers, daemons, or other computer-readable programs to be developed for a variety of applications of a flexible oversuits. Further, by providing encapsulation of functions of a flexible oversuit in a generic, accessible manner (e.g., by specifying and implementing an application programming interface (API) or other interface standard), computer-readable programs can be created to interface with the generic, encapsulated functions such that the computer-readable programs can enable operating modes or functions for a variety of differently-configured oversuit, rather than for a single type or model of flexible oversuit. For example, a virtual avatar communications program can access information about the posture of a wearer of a flexible oversuit by accessing a standard oversuit API. Differently-configured oversuits can include different sensors, actuators, and other elements, but can provide posture information in the same format according to the API. Other functions and features of a flexible oversuit, or other robotic, exoskeletal, assistive, haptic, or other mechatronic system, can be encapsulated by APIs or according to some other standardized computer access and control interface scheme.

FIG. 15 is a schematic illustrating elements of a oversuit 1900 and a hierarchy of control or operating the oversuit 1900. The flexible oversuit includes actuators 1920 and sensors 1930 configured to apply forces and/or torques to and detect one or more properties of, respectively, the oversuit 1900, a wearer of the oversuit 1900, and/or the environment of the wearer. The oversuit 1900 additionally includes a controller 1916 configured to operate the actuators 1920 and sensors 1930 by using hardware interface electronics 1940. The hardware electronics interface 1940 includes electronics configured to interface signals from and to the controller 1916 with signals used to operate the actuators 1920 and sensors 1930. For example, the actuators 1920 can include exotendons, and the hardware interface electronics 1940 can include voltage generators, switches, and capacitance meters to clutch and un-clutch the exotendons and to report the length of the exotendons. The hardware interface electronics 1940 can include voltage regulators, voltage generators, amplifiers, current detectors, encoders, magnetometers, switches, controlled-current sources, DACs, ADCs, feedback controllers, brushless motor controllers, or other electronic and mechatronic elements.

The controller 1916 additionally operates a user interface 1950 that is configured to present information to a user and/or wearer of the oversuit 1900 and a communications interface 1960 that is configured to facilitate the transfer of information between the controller 1916 and some other system (e.g., by transmitting a wireless signal). Additionally, or alternatively, the user interface 1950 can be part of a separate system that is configured to transmit and receive user interface information to/from the controller 1916 using the communications interface 1960 (e.g., the user interface 1950 can be part of a cellphone).

The controller 1916 is configured to execute computer-readable programs describing functions of the flexible oversuit 1912. Among the computer-readable programs executed by the controller 1916 are an operating system 1912, applications 1914a, 1914b, and 1914c, and calibration service 1914d. The operating system 1912 manages hardware resources of the controller 1916 (e.g., I/O ports, registers, timers, interrupts, peripherals, memory management units, serial and/or parallel communications units) and, by extension, manages the hardware resources of the oversuit 1900. The operating system 1912 is the only computer-readable program executed by the controller 1916 that has direct access to the hardware interface electronics 1940 and, by extension, the actuators 1920 and sensors 1930 of the oversuit 1900.

The applications 1914a, 1914b, 1914c are computer-readable programs that describe some function, functions, operating mode, or operating modes of the oversuit 1900. For example, application 1914a can describe a process for transmitting information about the wearer's posture to update a virtual avatar of the wearer that includes accessing information on a wearer's posture from the operating system 1912, maintaining communications with a remote system using the communications interface 1960, formatting the posture information, and sending the posture information to the remote system. The calibration service 1914d is a computer-readable program describing processes to store parameters describing properties of wearers, actuators 1920, and/or sensors 1930 of the oversuit 1900, to update those parameters based on operation of the actuators 1920, and/or sensors 1930 when a wearer is using the oversuit 1900, to make the parameters available to the operating system 1912 and/or applications 1914a, 1914b, 1914c, and other functions relating to the parameters. Note that applications 1914a, 1914b, 1914 and calibration service 1914d are intended as examples of computer-readable programs that can be run by the operating system 1912 of the controller 1916 to enable functions or operating modes of a oversuit 1900.

The operating system 1912 can provide for low-level control and maintenance of the hardware (e.g., 1920, 1930, 1940). In some examples, the operating system 1912 and/or hardware interface electronics 1940 can detect information about the oversuit 1900, the wearer, and/or the wearer's environment from one or more sensors 1930 at a constant specified rate. The operating system 1912 can generate an estimate of one or more states or properties of the oversuit 1900 or components thereof using the detected information. The operating system 1912 can update the generated estimate at the same rate as the constant specified rate or at a lower rate. The generated estimate can be generated from the detected information using a filter to remove noise, generate an estimate of an indirectly-detected property, or according to some other application. For example, the operating system 1912 can generate the estimate from the detected information using a Kalman filter to remove noise and to generate an estimate of a single directly or indirectly measured property of the oversuit 1900, the wearer, and/or the wearer's environment using more than one sensor. In some examples, the operating system can determine information about the wearer and/or oversuit 1900 based on detected information from multiple points in time. For example, the operating system 1900 can determine an eversion stretch and dorsiflexion stretch.

In some examples, the operating system 1912 and/or hardware interface electronics 1940 can operate and/or provide services related to operation of the actuators 1920. That is, in case where operation of the actuators 1920 requires the generation of control signals over a period of time, knowledge about a state or states of the actuators 1920, or other considerations, the operating system 1912 and/or hardware interface electronics 1940 can translate simple commands to operate the actuators 1920 (e.g., a command to generate a specified level of force using a twisted string actuator (TSA) of the actuators 1920) into the complex and/or state-based commands to the hardware interface electronics 1940 and/or actuators 1920 necessary to effect the simple command (e.g., a sequence of currents applied to windings of a motor of a TSA, based on a starting position of a rotor determined and stored by the operating system 1916, a relative position of the motor detected using an encoder, and a force generated by the TSA detected using a load cell).

In some examples, the operating system 1912 can further encapsulate the operation of the oversuit 1900 by translating a system-level simple command (e.g., a commanded level of force tension applied to the footplate) into commands for multiple actuators, according to the configuration of the oversuit 1900. This encapsulation can enable the creation of general-purpose applications that can effect a function of an oversuit (e.g., allowing a wearer of the oversuit to stretch his foot) without being configured to operate a specific model or type of oversuit (e.g., by being configured to generate a simple force production profile that the operating system 1912 and hardware interface electronics 1940 can translate into actuator commands sufficient to cause the actuators 1920 to apply the commanded force production profile to the footplate).

The operating system 1912 can act as a standard, multi-purpose platform to enable the use of a variety of oversuits having a variety of different hardware configurations to enable a variety of mechatronic, biomedical, human interface, training, rehabilitative, communications, and other applications. The operating system 1912 can make sensors 1930, actuators 1920, or other elements or functions of the oversuit 1900 available to remote systems in communication with the oversuit 1900 (e.g., using the communications interface 1960) and/or a variety of applications, daemons, services, or other computer-readable programs being executed by operating system 1912. The operating system 1912 can make the actuators, sensors, or other elements or functions available in a standard way (e.g., through an API, communications protocol, or other programmatic interface) such that applications, daemons, services, or other computer-readable programs can be created to be installed on, executed by, and operated to enable functions or operating modes of a variety of flexible oversuits having a variety of different configurations. The API, communications protocol, or other programmatic interface made available by the operating system 1912 can encapsulate, translate, or otherwise abstract the operation of the oversuit 1900 to enable the creation of such computer-readable programs that are able to operate to enable functions of a wide variety of differently-configured flexible oversuits.

Additionally or alternatively, the operating system 1912 can be configured to operate a modular flexible oversuit system (i.e., a flexible oversuit system wherein actuators, sensors, or other elements can be added or subtracted from a flexible oversuit to enable operating modes or functions of the flexible oversuit). In some examples, the operating system 1912 can determine the hardware configuration of the oversuit 1900 dynamically and can adjust the operation of the oversuit 1900 relative to the determined current hardware configuration of the oversuit 1900. This operation can be performed in a way that was ‘invisible’ to computer-readable programs (e.g., 1914a, 1914b, 1914c) accessing the functionality of the oversuit 1900 through a standardized programmatic interface presented by the operating system 1912. For example, the computer-readable program can indicate to the operating system 1912, through the standardized programmatic interface, that a specified level of torque was to be applied to an ankle of a wearer of the oversuit 1900. The operating system 1912 can responsively determine a pattern of operation of the actuators 1920, based on the determined hardware configuration of the oversuit 1900, sufficient to apply the specified level of torque to the ankle of the wearer.

In some examples, the operating system 1912 and/or hardware interface electronics 1940 can operate the actuators 1920 to ensure that the oversuit 1900 does not operate to directly cause the wearer to be injured and/or elements of the oversuit 1900 to be damaged. In some examples, this can include not operating the actuators 1920 to apply forces and/or torques to the body of the wearer that exceeded some maximum threshold. This can be implemented as a watchdog process or some other computer-readable program that can be configured (when executed by the controller 1916) to monitor the forces being applied by the actuators 1920 (e.g., by monitoring commands sent to the actuators 1920 and/or monitoring measurements of forces or other properties detected using the sensors 1930) and to disable and/or change the operation of the actuators 1920 to prevent injury of the wearer. Additionally or alternatively, the hardware interface electronics 1940 can be configured to include circuitry to prevent excessive forces and/or torques from being applied to the wearer (e.g., by channeling to a comparator the output of a load cell that is configured to measure the force generated by a TSA, and configuring the comparator to cut the power to the motor of the TSA when the force exceeded a specified level).

In some examples, operating the actuators 1920 to ensure that the oversuit 1900 does not damage itself can include a watchdog process or circuitry configured to prevent over-current, over-load, over-rotation, or other conditions from occurring that can result in damage to elements of the oversuit 1900. For example, the hardware interface electronics 1940 can include a metal oxide varistor, breaker, shunt diode, or other element configured to limit the voltage and/or current applied to a winding of a motor.

Note that the above functions described as being enabled by the operating system 1912 can additionally or alternatively be implemented by applications 1914a, 1914b, 1914c, services, drivers, daemons, or other computer-readable programs executed by the controller 1900. The applications, drivers, services, daemons, or other computer-readable programs can have special security privileges or other properties to facilitate their use to enable the above functions.

The operating system 1912 can encapsulate the functions of the hardware interface electronics 1940, actuators 1920, and sensors 1930 for use by other computer-readable programs (e.g., applications 1914a, 1914b, and 1914c, calibration service 1914d), by the user (through the user interface 1950), and/or by some other system (i.e., a system configured to communicate with the controller 1916 through the communications interface 1960). The encapsulation of functions of the oversuit 1900 can take the form of application programming interfaces (APIs), i.e., sets of function calls and procedures that an application running on the controller 1916 can use to access the functionality of elements of the oversuit 1900. In some examples, the operating system 1912 can make available a standard ‘oversuit API’ to applications being executed by the controller 1916. The ‘oversuit API’ can enable applications 1914a, 1914b, 1914c to access functions of the oversuit 1900 without requiring those applications 1914a, 1914b, 1914c to be configured to generate whatever complex, time-dependent signals are necessary to operate elements of the oversuit 1900 (e.g., actuators 1920, sensors 1930).

The ‘oversuit API’ can allow applications 1914a, 1914b, 1914c to send simple commands to the operating system 1912 (e.g., ‘begin storing mechanical energy from the ankle of the wearer when the foot of the wearer contacts the ground’) in such that the operating system 1912 can interpret those commands and generate the command signals to the hardware interface electronics 1940 or other elements of the oversuit 1900 that are sufficient to effect the simple commands generated by the applications 1914a, 1914b, 1914c (e.g., determining whether the foot of the wearer has contacted the ground based on information detected by the sensors 1930, responsively applying high voltage to an exotendon that crosses the user's ankle).

The ‘oversuit API’ can be an industry standard (e.g., an ISO standard), a proprietary standard, an open-source standard, or otherwise made available to individuals that can then produce applications for oversuits. The ‘oversuit API’ can allow applications, drivers, services, daemons, or other computer-readable programs to be created that are able to operate a variety of different types and configurations of oversuits by being configured to interface with the standard ‘oversuit API’ that is implemented by the variety of different types and configurations of oversuits. Additionally or alternatively, the oversuit API′ can provide a standard encapsulation of individual oversuit-specific actuators (i.e., actuators that apply forces to specific body segments, where differently-configured oversuits may not include an actuator that applies forces to the same specific body segments) and can provide a standard interface for accessing information on the configuration of whatever oversuit is providing the ‘oversuit API’. An application or other program that accesses the oversuit API′ can access data about the configuration of the oversuit (e.g., locations and forces between body segments generated by actuators, specifications of actuators, locations and specifications of sensors) and can generate simple commands for individual actuators (e.g., generate a force of 30 newtons for 50 milliseconds) based on a model of the oversuit generated by the application and based on the information on the accessed data about the configuration of the oversuit. Additional or alternate functionality can be encapsulated by an oversuit API′ according to an application.

Applications 1914a, 1914b, 1914c can individually enable all or parts of the functions and operating modes of a flexible oversuit described herein. For example, an application can enable haptic control of a robotic system by transmitting postures, forces, torques, and other information about the activity of a wearer of the oversuit 1900 and by translating received forces and torques from the robotic system into haptic feedback applied to the wearer (i.e., forces and torques applied to the body of the wearer by actuators 1920 and/or haptic feedback elements). In another example, an application can enable a wearer to locomote more efficiently by submitting commands to and receiving data from the operating system 1912 (e.g., through an API) such that actuators 1920 of the oversuit 1900 assist the movement of the user, extract negative work from phases of the wearer's locomotion and inject the stored work to other phases of the wearer's locomotion, or other methods of operating the oversuit 1900. Applications can be installed on the controller 1916 and/or on a computer-readable storage medium included in the oversuit 1900 by a variety of methods. Applications can be installed from a removable computer-readable storage medium or from a system in communication with the controller 1916 through the communications interface 1960. In some examples, the applications can be installed from a web site, a repository of compiled or un-compiled programs on the Internet, an online store (e.g., Google Play, iTunes App Store), or some other source. Further, functions of the applications can be contingent upon the controller 1916 being in continuous or periodic communication with a remote system (e.g., to receive updates, authenticate the application, to provide information about current environmental conditions).

The oversuit 1900 illustrated in FIG. 15 is intended as an illustrative example. Other configurations of flexible oversuits and of operating systems, kernels, applications, drivers, services, daemons, or other computer-readable programs are anticipated. For example, an operating system configured to operate an oversuit can include a real-time operating system component configured to generate low-level commands to operate elements of the oversuit and a non-real-time component to enable less time-sensitive functions, like a clock on a user interface, updating computer-readable programs stored in the oversuit, or other functions. A oversuit can include more than one controller; further, some of those controllers can be configured to execute real-time applications, operating systems, drivers, or other computer-readable programs (e.g., those controllers were configured to have very short interrupt servicing routines, very fast thread switching, or other properties and functions relating to latency-sensitive computations) while other controllers are configured to enable less time-sensitive functions of a flexible oversuit. Additional configurations and operating modes of an oversuit are anticipated. Further, control systems configured as described herein can additionally or alternatively be configured to enable the operation of devices and systems other than oversuit; for example, control systems as described herein can be configured to operate robots, rigid oversuits or exoskeletons, assistive devices, prosthetics, or other mechatronic devices.

Control of actuators of an oversuit can be implemented in a variety of ways according to a variety of control schemes. Generally, one or more hardware and/or software controllers can receive information about the state of the flexible oversuit, a wearer of the oversuit, and/or the environment of the oversuit from sensors disposed on or within the oversuit and/or a remote system in communication with the oversuit. The one or more hardware and/or software controllers can then generate a control output that can be executed by actuators of the oversuit to affect a commanded state of the oversuit and/or to enable some other application. One or more software controllers can be implemented as part of an operating system, kernel, driver, application, service, daemon, or other computer-readable program executed by a processor included in the oversuit.

In some embodiments, a powered assistive oversuit intended primarily for assistive functions can also be adapted to perform oversuit functions. In one embodiment, an assistive oversuit similar to the embodiments described in U.S. Publication No. 2018/0056104, that is used for assistive functions may be adapted to perform oversuit functions. Embodiments of such an assistive oversuit typically include FLAs approximating muscle groups such as hip flexors, gluteal/hip extensors, spinal extensors, or abdominal muscles. In the assistive modes of these oversuits, these FLAs provide assistance for activities such as moving between standing and seated positions, walking, and postural stability. Actuation of specific FLAs within such an oversuit system may also provide stretching assistance. Typically, activation of one or more FLAs approximating a muscle group can stretch the antagonist muscles. For example, activation of one or more FLAs approximating the abdominal muscles might stretch the spinal extensors, or activation of one or more FLAs approximating gluteal/hip extensor muscles can stretch the hip flexors. The oversuit may be adapted to detect when the wearer is ready to initiate a stretch and perform an automated stretching regimen; or the wearer may indicate to the suit to initiate a stretching regimen.

It can be appreciated that assistive oversuits may have multiple applications. Assistive oversuits may be prescribed for medical applications. These may include therapeutic applications, such as assistance with exercise or stretching regimens for rehabilitation, disease mitigation or other therapeutic purposes. Mobility-assistance devices such as wheelchairs, walkers, crutches and scooters are often prescribed for individuals with mobility impairments. Likewise, an assistive oversuit may be prescribed for mobility assistance for patients with mobility impairments. Compared with mobility assistance devices such as wheelchairs, walkers, crutches and scooters, an assistive oversuit may be less bulky, more visually appealing, and conform with activities of daily living such as riding in vehicles, attending community or social functions, using the toilet, and common household activities.

An assistive oversuit may additionally function as primary apparel, fashion items or accessories. The oversuit may be stylized for desired visual appearance. The stylized design may reinforce visual perception of the assistance that the oversuit is intended to provide. For example, an assistive oversuit intended to assist with torso and upper body activities may present a visual appearance of a muscular torso and upper body. Alternatively, the stylized design may be intended to mask or camouflage the functionality of the assistive oversuit through design of the base layer, electro/mechanical integration or other design factors.

Similarly to assistive oversuits intended for medically prescribed mobility assistance, assistive oversuits may be developed and utilized for non-medical mobility assistance, performance enhancement and support. For many, independent aging is associated with greater quality of life, however activities may become more limited with time due to normal aging processes. An assistive oversuit may enable aging individuals living independently to electively enhance their abilities and activities. For example, gait or walking assistance could enable individuals to maintain routines such as social walking or golf. Postural assistance may render social situations more comfortable, with less fatigue. Assistance with transitioning between seated and standing positions may reduce fatigue, increase confidence, and reduce the risk of falls. These types of assistance, while not explicitly medical in nature, may enable more fulfilling, independent living during aging processes.

Athletic applications for an assistive oversuit are also envisioned. In one example, an oversuit may be optimized to assist with a particular activity, such as cycling. In the cycling example, FLAs approximating gluteal or hip extensor muscles may be integrated into bicycle clothing, providing assistance with pedaling. The assistance could be varied based on terrain, fatigue level or strength of the wearer, or other factors. The assistance provided may enable increased performance, injury avoidance, or maintenance of performance in the case of injury or aging. It can be appreciated that assistive oversuits could be optimized to assist with the demands of other sports such as running, jumping, swimming, skiing, or other activities. An athletic assistive oversuit may also be optimized for training in a particular sport or activity. Assistive oversuits may guide the wearer in proper form or technique, such as a golf swing, running stride, skiing form, swimming stroke, or other components of sports or activities. Assistive oversuits may also provide resistance for strength or endurance training. The provided resistance may be according to a regimen, such as high intensity intervals.

Assistive oversuit systems as described above may also be used in gaming applications. Motions of the wearer, detected by the suit, may be incorporated as a game controller system.

Assistive oversuits as described above may be used for military or first responder applications. Military and first responder personnel are often to be required to perform arduous work where safety or even life may be at stake. An assistive oversuit may provide additional strength or endurance as required for these occupations. An assistive oversuit may connect to one or more communication networks to provide communication services for the wearer, as well as remote monitoring of the suit or wearer.

Assistive oversuits as described above may be used for industrial or occupational safety applications. Oversuits may provide more strength or endurance for specific physical tasks such as lifting or carrying or repetitive tasks such as assembly line work. By providing physical assistance, assistive oversuits may also help avoid or prevent occupational injury due overexertion or repetitive stress.

Assistive oversuits as described above may also be configured as home accessories. Home accessory assistive oversuits may assist with household tasks such as cleaning or yard work, or may be used for recreational or exercise purposes. The communication capabilities of an assistive oversuit may connect to a home network for communication, entertainment or safety monitoring purposes.

It is to be understood that the disclosed subject matter is not limited in its application to the details of construction and to the arrangements of the components set forth in the following description or illustrated in the drawings. The disclosed subject matter is capable of other embodiments and of being practiced and carried out in various ways. Also, it is to be understood that the phraseology and terminology employed herein are for the purpose of description and should not be regarded as limiting.

As such, those skilled in the art can appreciate that the conception, upon which this disclosure is based, may readily be utilized as a basis for the designing of other structures, systems, methods and media for carrying out the several purposes of the disclosed subject matter.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

What is claimed is:

1. An oversuit system comprising:
a fabric suit component comprising an upper portion constructed to be worn over an upper body region of a user and a lower portion constructed to be worn over a lower body region of the user,
wherein the upper portion comprises:
upper portion sensors;
a core support member comprising a core twisted string;
a trunk flexor tunnel comprising a trunk flexor twisted string; and
a trunk extensor tunnel comprising a trunk extensor twisted string; and
wherein the lower portion comprises:
lower portion sensors;
right knee load distribution member (LDM);
left knee LDM;
right flexor tunnel comprising a right flexor twisted string that is connected to the right knee LDM;
left flexor tunnel comprising a left flexor twisted string that is connected to the left knee LDM;
right extensor tunnel comprising a right extensor twisted string that is connected to the right knee LDM; and
left extensor tunnel comprising a left extensor twisted string that is connected to the left knee LDM; and
a belt member constructed to be worn over the fabric suit and around a hip region of the user, the belt member comprising:
a plurality of flexible linear actuators (FLA) constructed to be removably connected to designated twisted strings comprising the core twisted string, the trunk flexor twisted string, the trunk extensor twisted string, the right flexor twisted string, the left flexor twisted string, the right extensor twisted string, and the left extensor twisted string;
a power source; and
a controller operative to:
process data received by the upper portion sensors and the lower portion sensors to identify one of a plurality of static positions being held by the user; and
activate a subset of the plurality of FLAs based on the identified static position such that the oversuit supports the user in maintaining the identified static position.

2. The system of claim 1, wherein the plurality of static positions comprise standing, sitting, leaning forward, leaning backward, squatting, stooping, and kneeling.

3. The system of claim 1, wherein the upper portion sensors comprise first inertial measurement unit (IMU) and second IMU, and wherein the lower portion sensors comprise third IMU, fourth IMU, first pressure sensor, and second pressure sensor.

4. The system of claim 1, wherein the first IMU and the second IMU are positioned along a common axis with respect to each other, wherein third IMU is mounted in a right thigh region of the lower portion, wherein the fourth IMU is mounted in a left thigh region of the lower portion, wherein the first pressure sensor is mounted in the right knee LDM, and wherein the second pressure sensor is mounted in the left knee LDM.

5. The system of claim 1, wherein trunk flexor tunnel and the trunk extensor tunnel each have a line of action routing configuration that yields a 2:1 purchase ratio.

6. The system of claim 1, wherein the right flexor tunnel, the left flexor tunnel, the right extensor tunnel, and the left extensor tunnel each have a line of action routing configuration that yields a 3:1 purchase ratio.

7. The system of claim 1, wherein the trunk flexor tunnel, the trunk extensor tunnel, the right flexor tunnel, the left flexor tunnel, the right extensor tunnel, and the left extensor tunnel each comprise an attachment point and at least one re-routing structure.

8. The system of claim 7, wherein the at least one re-routing structure comprises a curved hollow tube.

9. The system of claim 1, wherein the processor is further operative to:
process data received by the upper portion sensors and the lower portion sensors to identify one of a plurality of compound static positions being held by the user; and
activate a subset of the plurality of FLAs based on the identified compound static position such that the oversuit supports the user in maintaining the identified compound static position.

10. The system of claim 9, wherein the compound static positions comprise sitting with a forward lean, sitting with a backward lean, kneeling with a forward lean, and kneeling with a backward lean.

11. An oversuit system comprising:
a fabric suit component constructed to be worn over a body of a use, wherein the fabric suit component comprises:
sensors;
a core support member comprising a core twisted string;
a left knee load distribution member (LDM);
a right knee LDM;
a plurality of tunnels constructed to route twisted strings;
a belt member constructed to be worn over the fabric suit and around a hip region of the user, the belt member comprising:
a plurality of flexible linear actuators (FLA) constructed to be removably connected to designated twisted strings comprising twisted strings being routed through the plurality of tunnels and the core twisted string;
a power source; and
a controller operative to:
process data received by the sensors to identify one of a plurality of static positions being held by the user; and
activate a subset of the plurality of FLAs based on the identified static position such that the oversuit supports the user in maintaining the identified static position.

12. A method for using an oversuit comprising a fabric suit component and a belt, wherein the fabric suit comprises load distribution members and twisted stings that are connected to the load distribution members, and wherein the belt comprises flexible linear actuators that are connected to the twisted strings, the method comprising:
detecting a static position of a user of the oversuit; and
selectively activating a subset of the flexible linear actuators based on the detected static position such that the oversuit supports the user in maintaining the determined static position.
